(19) United States
(12) Patent Application Publication
(10) Pub. No.: US 2016/0202234 A1
(43) Pub. Date: Jul. 14, 2016

(54) METHOD FOR EVALUATING, VIA BIODOSIMETRY, THE IRRADIATION DOSE RECEIVED BY A PERSON SUBJECTED TO IONIZING RADIATION

(71) Applicant: ACUBENS, Antony (FR)

(72) Inventors: Ivana JUKIC, Paris (FR); Amélie SCHELLENBERG, Paris (FR); Jozo DELIC, Bourg-La-Reine (FR); Arnaud TUPINIER, Champigny Sur Marne (FR); Vincent PAGET, Chilly Mazarin (FR); Yohann FEDOR, Fontenay-aux-Roses (FR); Luc MORAT, Le Plessis Robinson (FR); Nicolas UGOLIN, Paris (FR); Caroline FLACK, Bagneus (FR); Sylvie CHEVILLARD, Paris (FR); Emilie LEFEVRE, Mennecy (FR)

(73) Assignee: ACUBENS, Antony (FR)

(21) Appl. No.: 14/777,724

(22) PCT Filed: May 15, 2014
(86) PCT No.: PCT/FR2014/051136

§ 371 Date: Sep. 16, 2015

(30) Foreign Application Priority Data
May 15, 2013 (FR) 1354347

(51) Int. Cl. G01N 33/483

(52) U.S. Cl. CPC G01N 33/4833; G01N 2800/40

(57) ABSTRACT

The invention relates to a method for evaluating, via biodosimetry, the irradiation dose received by a person subjected to ionizing radiation. Said method comprises: a) sampling, in at least one area of the body of the person, the follicles or bulbs of head and/or body hair of the person; b) extracting proteins from cells of said sampled hair bulbs or follicles, wherein said proteins, including proteins from the ATM system, are subjected to phosphorylation and/or acetylation induced by ionizing radiation; and c) analyzing at least two types of extracted proteins and interpreting the analysis results in order to determine the irradiation dose in the or each sampling area.

### TECHNICAL FIELD

The present invention relates to a method for evaluating, via biodosimetry, the irradiation dose received by a person subjected to ionising radiation.

### PRIOR ART

In the case of irradiations of the electromagnetic type, during an accident or an attack for example, there exist various means for determining the dose received by an individual (dosimetry), in particular through biological examinations in the laboratory.

It is possible for example to measure abnormalities at the chromosome level. This method requires culturing cells sampled from the individual, chromosome spreading and other highly technical manipulations. It is also possible to measure the lymphocyte apoptosis of the sampled cells. This method appears to be less expensive but requires the culturing of cells and an extremely tricky interpretation of the results. Other methods based on cell sorting have also been developed, in particular using the so-called FACS method (standing for fluorescence activated cell sorting) on blood cells. This intracell analysis method makes it possible to measure the level of biological markers (foci) that is proportional to the irradiation level received by the individual.

These approaches do however remain laboratory approaches that are complex and lengthy to implement. In general these laboratory methods are lengthy, complex and expensive and cannot be envisaged for tests on site or in the context of large-scale tests for a large number of persons, whether in the context of a sorting of persons or biodosimetry. In addition, these are global biodosimetry tests that give information on the global irradiation level of an individual. These tests are not effective in the case of heterogeneous irradiation where the individual has not received the same irradiation dose over the whole of the body. This is due to the fact that these tests are essentially based on measurements carried out on blood cells which, as is known, circulate throughout the body of an individual. Thus, with partial irradiations and/or low-dose irradiations (below 1 Gy), the measurement is diluted and the irradiation dose measured is therefore underestimated. It is furthermore impossible to identify the part of the body irradiated.

In the case of exposure to neutrons, there exist tests that can be used on site and which make it possible to estimate the neutron doses received. It is thus possible to measure the level of phosphorus-32 in the hair or sodium-24 in the blood. However, there also, these are global dosimetry tests.

All these methods are global dosimetry tests that do not make it possible to precisely identify the part or parts of the body irradiated. To envisage biodosimetry of a specific region of the body of an irradiated individual, it is currently necessary to use an invasive method consisting of carrying out skin biopsies in the region concerned. However, it is not possible to carry out multiple biopsies on the individual in order to seek all the irradiated regions of a body. In addition, the biopsies carried out create wounds that may be infected and are not compatible with an environment contaminated by radioactive particles. In effect, the requirements for sampling asepsia, and the highly corrosive products used for decontaminating persons, prohibit this type of practice on site. Finally, taking many samples makes the use of laboratory techniques accordingly more complex and further increases the cost of testing in the case of a large number of individuals liable to have been irradiated.

Methods for measuring DNA double-strand breaks in the cells of an irradiated individual can also be cited. This is because it is known that one of the major consequences for cells arising from ionising radiations is breaks in DNA and more particularly double-strand breaks (DSBs). It has thus been shown that the proportion of DNA double-strand breaks in an individual is directly proportional to the irradiation dose received by this individual. Measuring these breaks therefore makes it possible to determine the irradiation dose received by the individual.

DNA double-strand breaks, rapidly, and in a coordinated manner, cause a series of cell responses, including the activation of protein and enzyme systems for signalling and repairing damage to the DNA. This response in particular involves the proteins of the ATM system, also referred to as an ATM cascade. When the response systems of the cell are monopolised following double-strand breaks, certain proteins in the ATM system such as Chk2, NBS1, ATM, BRCA1, 53BP1, P53, KU70, etc. are phosphorylated whereas others are dephosphorylated. One of the proteins phosphorylated early in response to double-strand breaks is the histone H2AX. This histone is phosphorylated into γ-H2AX (Rogakou et al. (1998) J. Biol. Chem. 273: 5858-5868; Paull et al. (2000) Curr. Biol. 10:886-895). The accumulation of γ-H2AX at the double-strand breaks can be detected in immunofluorescence microscopy; it is a case of γ-H2AX foci or foci detected by the aforementioned FACS method. The number of foci in a cell is correlated with the number of double-strand breaks. It is therefore possible to carry out biodosimetry by measuring the number of foci in the cells (intracell measurement). Nevertheless, this approach is very lengthy, complex and expensive to implement. In addition, this method does not take into account the other proteins in the ATM system such as Chk2, NBS1, ATM, BRCA1, 53BP1, P53, KU70, etc. In addition, none of the methods described makes it possible to determine the irradiation dose received and the time at which it took place. This is in particular the case in the documents “The Use of Gamma-H2AX as a Biodosimeter for Total-Body Radiation Exposure in Non-Human Primates” by Christophe E. Redon et al., PLOS ONE, vol. 5, no. 11, 23 Nov. 2010, page e15544, and “Q_{γ-H2AX}, an analysis method for partial-body radiation exposure using γ-H2AX in non-human primate lymphocytes” by Christophe E. Redon et al., Radiation Measurements, Elsevier, Amsterdam, NL, vol. 46, no. 9, 25 Feb. 2011, pages 877-881.

There therefore exists a real need for a method that can be implemented quickly and that is especially but not exclusively suited to sites for identifying, sorting and where applicable quantifying the irradiation level in persons irradiated in the context of an accident or an attack involving irradiations of the ionising type.

The aim of the present invention is in particular to afford a simple, effective and economical solution to at least some of the aforementioned problems.

### DISCLOSURE OF THE INVENTION

The invention proposes a method for evaluating, via biodosimetry, the irradiation dose received by a person subjected to ionising radiation, comprising the steps consisting of:
a) sampling, in at least one region of the body of the individual, hair bulbs or follicles of bristles and/or hairs of the individual,
b) extracting proteins from cells of these hair bulbs or follicle samples, these proteins comprising proteins from the ATM system that have undergone phosphorylation and/or acetylation caused by the ionising radiation, and
c) analysing at least two types of protein extracted and interpreting the analysis results in order to determine the irradiation dose received in the or each sampling region, the phosphorylated and/or acetylated proteins from the ATM system being intended to produce signals during the analysis, a quantity of which, such as an intensity or an intensity ratio, makes it possible to evaluate this irradiation dose.

In the present application, biodosimetry means the quantitative determination of the dose absorbed by a cell, an organism or an individual following exposure to ionising radiations. The biodosimetry is here extracellular, that is to say the analysis of the measurement is done not inside the cell (in contradistinction to the FACS intracell method of the prior art), but directly on a mixture of proteins obtained after destruction of the plasma sand nucleus membranes of the cell (cell lysis). The irradiation dose received by an individual is in general expressed in gray (Gy).

In the present application, the terms and expressions “bristles”, “hairs”, “bristles and/or hairs” designate indifferently bristles and/or hairs. The terms and expressions “bulbs”, “hair follicles”, “hair bulbs and/or follicles” designate indifferently hair follicles and/or bulbs coming from bristles and/or hairs.

“Extracting proteins” means making proteins accessible to antibodies, markers or the like outside the cell.

Irradiation designates the intentional or accidental exposure of a cell, organism, substance or body to radiations, in particular to ionising radiations. An ionising radiation is radiation capable of depositing enough energy in the material that it passes through to create ionisation. The properties of ionising radiations depend in particular on the nature of the particles constituting the radiation as well as the energy thereof. They have various natures and sources, such as ultraviolet radiation, X-rays, neutrons, radioactive sources, and gamma, alpha, beta, etc. radiations. Ionising radiation reaching a living organism damages its cell constituents, such as the DNA. Both at low dose and at high doses, ionising radiations create double-strand breaks proportionally to the dose received. Intracell mechanisms may repair the lesions produced. On the other hand, in the case of exposure to high doses, these mechanisms are swamped and DNA double-strand breaks are not repaired, usually leading to cell death by apoptosis.

The ATM system or ATM cascade is an intracell mechanism in which some proteins undergo one or more phosphorylations, acetylations, dephosphorylations and/or deacetylations in response to a double-strand break in the DNA. This is because, following a DNA double-strand break, the ATM dimer protein is phosphorylated, causing the separation of the dimer into two monomers, which can then be fixed on target proteins.

Phosphorylation/dephosphorylation chain reactions of various substrates, such as the histone H2AX and the proteins 53BP1, BRCA1, Chk1 and Chk2, will cause stoppage of the cell cycle and activation of control points, which will allow repair of the damaged DNA or apoptosis of the cells if the DNA is irreparable.

Phosphorylation of a protein is the addition of a PO₃²⁻ phosphoryl group (more usually referred to as a phosphate group) that is transferred onto a protein, whereas on the other hand dephosphorylation is the loss of a phosphoryl group PO₃²⁻. In both cases, it is generally a case of frequent regulation mechanisms. Thus many enzymes and receivers are put in the “active” or “non-active” position by a phosphorylation or a dephosphorylation.

Likewise, acetylation of a protein is the transfer of an acetyl group (—C(O)CH₃) onto a protein. It generally permits activation or inhibition of enzymes.

As will be described in more detail hereinafter, analysing the results may consist in particular of quantifying the intensity of signals generated by the phosphorylated and/or acetylated proteins so as to deduce therefrom the irradiation dose received by the individual. This is because, the higher the irradiation dose received by the individual, the greater the number of phosphorylated/acetylated proteins, and the greater the intensity of the signals generated by these proteins during the analysis.

The steps of the method according to the invention are relatively simple to implement, which makes the method applicable particularly but not exclusively on site, for example at the place or close to the place where the irradiation source is situated. This method may also be applicable in a medical environment, in particular after or before an examination and/or treatment with X-rays. This is made possible by the nature of the samplings made on the individual. These samplings do not affect the health of the individual and are less painful than biopsies. They make it possible to have access to biological cells situated at particular regions of the body of the individual. The invention therefore makes it possible to make a rapid local measurement of irradiation (measurement of the irradiation dose received at the given region of the body of the individual), while the previous techniques allowed only a global measurement of irradiation (measurement of the irradiation dose received by the individual overall). The samplings may also be carried out simultaneously on a large number of individuals and over a relatively short period. The duration of implementation of the method may be around 20 minutes per individual, for example, and in general may be less than one hour. It is more particularly steps b) and c) of the method that can be implemented quickly. In the case where samplings are carried out on several different regions of the body of an individual, the method makes it possible to determine the irradiation dose received for each region and thus establish a body mapping of irradiation of the individual. The irradiation received is preferably gamma radiation.

The present invention thus makes it possible to determine whether an individual has been irradiated, as well as the level and extent of the irradiation of this individual and optionally to determine the moment of irradiation. The method makes it possible to have a rapid result so as to be able to quickly classify the individuals tested according to their irradiation level.

Advantageously, the method may be applied to several individuals, in a place normally not equipped with laboratory apparatus. It may also comprise a preliminary step of identifying the or each individual.

Said at least two types of protein analysed in step c) preferably have different phosphorylation/dephosphorylation kinetics. In other words, during the analysis, the signals corresponding to the two types of protein have different developments, which makes it possible in particular to date the irradiation. With an isolated observation of the signals related to two proteins, the level of each signal makes it possible, by means of a calibration curve, for example in three dimensions, defining the response according to the doses and time after exposure, to combine the information coming from the two proteins in order to define the dose received with respect to the time elapsed after irradiation. In a kinetic observation of the signals of the two proteins, for example through several samplings, each signal curve comprises two parts, a first part that increases (in which the intensity increases because of the increase in the degree of phosphorylation of the proteins), and a second part that decreases (the intensity decreases because of the dephosphorylation of the proteins in response to the repair of the DNA). The signal corresponding to the protein H2AX is different from those of the other proteins of the ATM system and in particular that of the protein Ku70. The signal from the protein H2AX in general comprises a first part that increases rapidly unlike that of the protein Ku70, which comprises a first part that increases more slowly. At a time t, even if the intensity of the signal of one of the types of protein decreases, the intensity of the signal of the other type is taken into account in order to increase the precision of the measurement. The development of the signal and its development speed (and the derivative of the curves or finite increment of the curves) give the corrective information for estimating the dose according to the individual radiosensitivity. The combination of the information from at least two markers makes it possible to define firstly, by observing at one point in time, the dose received and the time between the observation and the measurement, and secondly, in dynamic observation, the level of radiosensitivity.

In a preferred embodiment of the invention, the proteins analysed at step c) are at least H2AX and Ku70. Analysis of the phosphorylated form of Ku70 is advantageous since the inventors have found that the corresponding signal is more persistent than that of γ-H2AX and is reinforced for a longer period because the first part of its curve increases slowly unlike γ-H2AX.

In a preferential embodiment, the proteins are immobilised on a substrate at step c), the proteins then being fixed either directly, that is to say by interactions with the substrate, or indirectly, to said substrate. This indirect fixing can be achieved in a specific manner (by means of an antibody, a heptamer or any other molecule specifically recognising the protein or proteins of interest) or aspecifically (for example through DNA molecules fixed to the substrate). Fixing the proteins to a substrate makes it possible to facilitate and accelerate the analysis and therefore makes it possible to obtain results relatively quickly, for example in less than one hour. Fixing may be achieved chemically, for example by covalent bonding. Fixing can also be done on any substrate, such as a particle (micrometric, submicrometric or nanometric), a membrane (PVDF or nitrocellulose, or any other membrane able to capture the proteins directly or indirectly), or a filter for capturing the proteins. In a preferential embodiment, the substrates comprise diamond phases, a deposition of diamond or particles of diamond making it possible to capture the proteins directly or indirectly. In an even more preferential embodiment, these diamond phases will be hydrogenated so as to obtain C—H carbons capable of establishing covalent bonds through a carbamide.

In some embodiments, the substrate may comprise fragments of nucleic acid or analogue of additional nucleic acid complementary to attachment fragments of nucleic acid or analogue, fixed to the molecules capable of specifically recognising the proteins of interest which, by hybridising with said attachment fragments, allow fixing of the molecules capable of specifically recognising the proteins of interest on the substrate.

In some embodiments, the particulate substrates are paramagnetic or sensitive to a magnetic field.

In a particularly preferred embodiment, analysis step c) is carried out directly on the substrate on which the proteins extracted at step b) are fixed, which affords extremely rapid implementation of the method.

In a particular embodiment, the hair follicles are sampled in several distinct regions of the body, so as to determine the irradiation dose received by each region.

In a particular embodiment of the method according to the invention, sampling step a) comprises the substeps consisting of:
applying a patch to the or each region, and removing it in order to pull off hairs with their follicles in this region, and
introducing the patch and hairs with their follicles into a reaction well.

The patch may comprise a wax, an adhesive, a glue or any other sticky material making it possible to perform a depilation. After application of the patch to the sampling region, removal of the path pulls off the hairs with their follicles.

In the present application, well or reaction well means a receptacle in which the proteins of interest are extracted and can be isolated. A reaction well typically has a volume from a few microlitres to a few millilitres. Preferentially, the reaction well may be the well of a multiwell plate, such as a plate with 6, 12, 24, 48 or 96 wells. In the case of establishment of a body mapping of irradiation of an individual, each sample is introduced into a given well of a multiwell plate. Each well is preferably provided with a marking means for knowing with which sample it is associated.

According to a first embodiment of the invention, the patch is fixed to one end of a piston able to move in a tube between an advanced position and a retracted position. In the advanced position, the patch extends at least partly outside the tube and can be applied by means of the piston to the region to be depilated. In the retracted position, the patch is housed in the tube, and removing the patch from the region causes the hairs to be pulled off with their follicles. Retraction of the piston into the tube causes the follicles of the hairs attached to the patch to be introduced into the tube.

In a second embodiment of the invention, the patch is formed by a flexible strip that is fixed by one end to a rod carried by a plug, the strip being intended to be coiled around the rod before it is introduced into the reaction well. The hairs are preferably situated on the outside of the strip after coiling.

In a third embodiment, the patch is fixed to a membrane of flexible plastics material that allows application to a larger body surface area and therefore makes it possible to obtain a sample with more follicles. The flexible membrane can be intended to close the opening of the reaction wells, the wax or glue on the patch serving as an adhesive for closing these wells. The wax or glue on the patch preferably has a thickness greater than that of the hairs so that the patch can be glued without the hairs interfering with the gluing and its impermeability.

In the first embodiment, the retraction of the piston into the tube causes the folding of the hair follicles towards the inside of the tube. The folding facilitates the introduction of the sample hairs with their hair follicles in the reaction well. In the second embodiment, the patch is coiled around a rod in order to be able to be introduced easily into the reaction well. It is possible to keep the patch coiled by means of an adhesive disposed on the rod. In the third example, the patch is directly applied to the opening of a reaction well so as to close this opening with the patch, thus forming a closure film, the glue used serving as a closure adhesive. The three embodiments afford a not insignificant saving in time during tests carried out on a large number of individuals and on a large number of regions of the body of each individual. Thus, for a given individual, it is possible to carry out a precise biodosimetry test in less than one hour and to determine the irradiation dose received for each region of the body where a sampling has been carried out.

Advantageously, the patch is introduced into the well by fitting an open end of the aforementioned tube (from the first embodiment) in the well or by inserting the rod and strip (from the second embodiment) in the well and then closing this well with the plug carrying the rod.

In a particular embodiment, the method according to the invention further comprises the steps consisting of:
taking blood from the individual, for example at the end of a finger,
recovering cells from the blood taken, with the exception of the red corpuscles, and extracting proteins from these cells, these proteins comprising proteins of the ATM system that underwent phosphorylation and/or acetylation caused by the ionising radiation.

In the present application, the “cells” recovered from the sampled blood are blood cells from which the red corpuscles are excluded.

Analysing the blood cells makes it possible to determine the degree of injury of the genome DNA of the lymphocytes. This is a global measurement of the irradiation dose received by an individual.

Preferably, these steps precede step a) or are carried out simultaneously with steps a) and b). It is thus possible to carry out the sampling of blood and hairs simultaneously or almost simultaneously. It is also possible to carry out the steps of extracting proteins from the blood cells and hair follicles simultaneously or almost simultaneously. This affords a not insignificant saving in time.

It can however be entirely envisaged carrying out the aforementioned steps relating to the blood cells at any time in the method described above.

In a particular embodiment of the method according to the invention, the blood sampling step is preceded by a preliminary step consisting of applying a solution containing alcohol and heparin (or any mixture containing an antiseptic and an anticoagulant) to the sampling region, this sampling step being able to be followed by an additional step consisting of applying a solution containing alcohol and calcium chloride (or any mixture containing an antiseptic and a coagulant) to the sampling region.

The antiseptic may be chosen from alcohols, such as ethanol or isopropanol, or a mixture of disinfectants or antiseptics, and will preferably be an alcohol.

The anticoagulant may be heparin.

In a particular embodiment, the anticoagulant and the antiseptic are impregnated in a disposable wipe stored in an individual pack closed hermetically and quickly openable.

Similarly, the coagulant and the antiseptic can be impregnated in a disposable wipe stored in an individual pack closed hermetically and quickly openable.

The coagulant permits coagulation of the blood after sampling. This prevents any haemorrhage, in particular in the case of a haemophilic individual. The coagulant is preferably calcium chloride.

According to one embodiment, the blood sampling is carried out by means of a lancet intended to pierce the skin of the individual. This lancet comprises an ejectable blade and preferably comprises a chamber for storing a solution of alcohol and heparin (or any mixture containing an antiseptic and an anticoagulant such as oxalate, citrate, EDTA, heparin, etc.), which is intended to be released at the time of ejection of the blade.

The ejectable blade of the lancet is ejected and pierces the skin of the individual for the purpose of taking a blood sample. Preferably, the pricking will be carried out at the end of the finger. This lancet reduces the risks of contamination that might exist by sampling blood with a syringe, for example, since the sampling is less invasive and the wound smaller. In addition, such a lancet enables the individual to take the sample himself and thus to shorten the sampling period. It is possible to provide in the lancet a chamber for storing the solution containing the antiseptic and/or anticoagulant so that, when the blade, initially inside the lancet, is ejected, it pierces the chamber and releases its content. Preferably, this chamber will comprise a heparin solution for preventing coagulation of the blood and thus permit collection of a sufficient quantity of blood to perform the test.

It may be a disposable lancet, for example a lancet intended for diabetics.

According to an embodiment of the method according to the invention comprising a blood sampling step, the cell recovery step consists of:
introducing the sampled blood into a tube containing a suspension of magnetic balls functionalised with antibodies against blood group antigens, and optionally heparin (or any anticoagulant),
separating the balls from a residual liquid by means of a magnetic field, the residual liquid comprising the blood cells, with the exception of the red corpuscles, and transferring the residual liquid into a reaction well.

In the present application, the expression “residual liquid” designates a liquid comprising in particular plasma with lymphocytes and platelets, in particular the cells that are the subject of the test.

The magnetic balls functionalised with antibodies against blood group antigens make it possible to separate the red corpuscles from the cells of interest of the blood contained in the residual liquid, the red corpuscles being liable to generate undesirable signals complicating the interpretation of the measurements. The magnetic balls provided with antibodies are fixed to the red corpuscles in order to form clots. These clots are precipitated and retained by means of a magnet when the residual liquid is transferred into the reaction well. An anticoagulant such as heparin may be added to the sampled blood and/or to the reaction well.

In another embodiment, the balls comprise antibodies solely directed against the lymphocytes, so as to capture the latter and separate them from the other elements concerned of the blood, the proteins then being extracted on the lymphocyte cells precipitated by the balls.

According to an embodiment of the method according to the invention, the extraction of the proteins comprises the substeps consisting of:
putting the sampled hair follicles and/or the recovered residual liquid in contact with, or mixing them with, a cell lysis solution, or
putting the cells in contact with, or mixing them with, a non-denaturing solution, preferably free from phosphorus, and then subjecting the whole to an electromagnetic or mechanical energy, such as microwaves or ultrasound, intended to cause lysis of the cells.

Cell lysis permits destruction of the plasmic and nuclear membrane of the cell and extracts the proteins present therein. It can be carried out chemically, mechanically or by means of waves (microwaves, ultrasound, etc.). In the case of the chemical method a cell lysis solution is introduced into the reaction well. The cell lysis solution may comprise either a hypotonic solution (NaCl concentration<150 mM), or a hypertonic solution (NaCl concentration>150 mM) with an addition of enzymatic detergents (0.5-1% NP40, 0.1-1% SDS, Triton, RIPA, etc.). In the case of a mechanical cell lysis or one by means of waves, the cells may be put in contact or mixed with a non-denaturing solution. They are then subjected to a mechanical lysis caused for example by a press, glass balls or magnetic balls (moved by a magnetic field), ultrasound, etc. In a preferential embodiment, the lysis is carried out by means of waves, for example microwaves. Preferably, the non-denaturing solution is free from phosphorus in order not to subsequently disturb the interpretation of the results, in particular during an LIBS analysis. It may be a TBS (Tris-buffered saline) or TBE (Tris-buffered EDTA) solution. The cell lysis is preferably carried out by microwaves, which makes it possible not to modify the phosphorylation state of the proteins.

Advantageously, the various samples are distributed in various reaction wells of a multiwell plate. In the case of a large number of samples, this facilitates the various steps following sampling, in particular making it possible to automate them. One multiwell plate may be allocated to each individual tested. The wells of this plate comprise samples from several distinct regions of the body of the individual.

In an embodiment using a multiwell plate, the bottom of the wells may be formed by a membrane, optionally a filtering membrane, on which the proteins are intended to be deposited or fixed. They may be fixed to the bottom directly or by means of specific antibodies previously fixed to the bottom or to particles. In a preferential embodiment, the bottom of the wells is removable.

In one embodiment of the invention, markers are introduced into the reaction wells. These markers are fluorescent, colorimetric or chemiluminescent markers, and/or antibodies, and are intended to be bonded to phosphorylated and/or non-phosphorylated proteins.

In one embodiment of the invention, antibodies intended to fix the proteins being researched are fixed to particles that are introduced into the reaction wells. In one even more particular embodiment, each type of antibody is fixed to a particle of given size and which is different from the sizes of the particles carrying the other types of marker.

In one embodiment of the invention, the bottom of each reaction well comprises an immunochromatography test. It may for example be a strip of nitrocellulose allowing a mobile phase to migrate. The mobile phase comprises marked antibody/target protein complexes, proteins, and marked antibodies. The antibodies are marked for example by gold particles or balls. In this embodiment, the strip comprises, in a deposition region, marked antibodies directed against the phosphorylated epitope of at least one target protein of the mobile phase. These antibodies are not fixed to the strip and can be freeze-dried. The strip also comprises at least one region comprising antibodies directed against non-phosphorylated epitopes of at least one target protein. These antibodies are fixed to the strip and form the fixed phase. The strip may also comprise at least one region directed against the antibodies of the mobile phase or against the marking of these antibodies. When the sample of extracted proteins is deposited on the deposition region of the strip, the target proteins combine in a complex with the specific marked antibodies of the mobile phase. Hydration of the strip allows the mobile phase (marked antibody/target protein complexes, proteins and marked antibodies) to migrate into the nitrocellulose strip. By passing into the regions comprising the fixed phases, the marked antibody/phosphorylated target protein complexes and the non-phosphorylated target proteins are retained at the antibodies of the fixed phase. The intensity of the marking at the various fixed phases indicates the level of phosphorylated proteins. Several successive regions comprising a different fixed phase may be disposed in order to retain various types of target protein. The last region will comprise a fixed phase capturing the marked antibodies of the mobile phase that have not reacted. The ratio between the intensity of the marking of the fixed phase that has captured the target proteins and the intensity of the marking of the fixed phase that has captured the antibodies that have not reacted makes it possible to determine the number of targets.

In some embodiments, a known quantity of at least one marked target protein (reference protein) is introduced into the protein extract to be analysed. This marked protein competes with the complexing of the target protein in the corresponding fixed phase in order to obtain a signal of the sample (Iₛₐₘₚₗₑ). The same quantity of the marked target protein is introduced into another reaction well in order to obtain a reference signal (I_{ref}). Comparing the intensities of the signals I_{ref} and I obtained between the well comprising the referenced protein alone and the well comprising the reference protein mixed with the extract of proteins to be analysed makes it possible to standardise the phosphorylation measurements from a non-marked protein extract, so that the retained signal is Iₛₐₘₚₗₑ/I_{ref} or I_{ref}/I.

In some embodiments, the antibodies of the mobile phase and of the fixed phases may be reversed, that is to say the antibodies of the mobile phase are against the phosphorylated forms and the antibodies of the fixed phase are against the phosphorylated forms.

In some other embodiments, the antibodies may be marked by markers other than gold particles, such as fluorescent, colorimetric or chemiluminescent markers.

Likewise the target proteins may be marked differently by fluorescent, colorimetric or chemiluminescent markers.

In one embodiment of the invention, the analysis and interpretation step c) comprises the substeps consisting of:
subjecting each type of protein to a marking, the phosphorylated form of each type of protein optionally being able to have a specific marking,
subjecting the proteins to an analysis in which at least one signal caused by the marking of the protein type is studied,
comparing a quantity, such as the intensity, of this signal with a calibration curve representing the change in this quantity as a function of an irradiation dose received by an individual, and
deducing therefrom the irradiation dose received by the individual, in the sampling region.

Advantageously, in the above analysis substep, the proteins are analysed:
by an LIBS method that makes it possible to quantify at least the phosphorylation, each type of protein, in its phosphorylated and non-phosphorylated forms, optionally being able to be subjected to a marking, for example with boron, or
by fluorescence, colorimetry or chemiluminescence, each type of protein having been subjected to a fluorescent, colorimetric or chemiluminescent marking.

A predetermined quantity of radiomimetic chemotherapeutic substance may be added to the proteins extracted, and reference is then made to a treated sample.

A predetermined quantity of radiomimetic chemotherapeutic substance may be added to the proteins extracted, and the parameter studied is then:
IP: the intensity of at least one signal corresponding to the phosphorylated form of one of each type of protein, and/or
IP/I, the ratio between the intensity IP and the intensity I, I being the intensity of at least one signal corresponding to the marking of each type of protein, in its phosphorylated and non-phosphorylated forms, and/or
IP/IP_{treated}: the ratio between the intensity IP and the intensity IP_{treated}, IP_{treated} being the intensity of at least one signal corresponding to the phosphorylated and treated form of one of each type of protein, and/or
IP_{treated}/I_{treated} the ratio between the intensity IP_{treated} and the intensity I_{treated}, I_{treated} being the intensity of the signal corresponding to the marking of each type of treated protein, in its phosphorylated and non-phosphorylated forms, and/or
(IP/I)/(IP_{treated}/I_{treated}): the ratio between the intensities IP and I and the ratio between the intensities IP_{treated} and I_{treated}.

In one embodiment of the invention, step a) consists of sampling hair follicles on at least two occasions in the same sampling region.

Advantageously, the samplings are carried out at a predetermined interval of time Δt or are carried out almost simultaneously and at least one of the samples is cultured for a given period Δt.

The effects of the irradiation are immediate but decline over time. This is because the mechanism of repair of the DNA double-strand breaks is established as from the irradiation and differently for each individual. For the same individual, the degree of phosphorylation will then not be the same at time t=0 and at time Δt. It is therefore useful to know the rate of repair of the DNA double-strand breaks for each individual. This double sampling thus makes it possible to determine the rate of repair of the double-strand breaks for each individual and to take it into account to give more precision in the measurement of the irradiation dose received. This also makes it possible to determine the radiosusceptibility and the interindividual variation of each individual. The period Δt may be between 1 minute and 12 hours.

The radiosensitivity or radiosusceptibility of an individual corresponds to the sensitivity of the living tissues to ionising radiation, in particular the speed at which the individual repairs his DNA at the moment when the measurement is made. It varies from one individual to another, but also over time for a given individual.

The interindividual variation corresponds here to the speed of repair of the double-strand break of a given individual.

In an embodiment of the invention comprising at least two samplings spaced apart by a period Δt, the quantity or parameter studied is:
IP: the intensity of at least one signal corresponding to the phosphorylated form of one of each type of protein, and/or
IP/I: the ratio between the intensities IP and I, I being the intensity of at least one signal corresponding to the marking of this type of protein, in its phosphorylated and non-phosphorylated forms, and/or
dIP/dt: the variation over time in the intensity IP, and/or
d(IP/I)/dt: the variation over time in the ratio IP/I.

In an embodiment of the invention comprising at least two samplings of hair follicles, at least two of the samples are mixed with a radiomimetic chemotherapeutic substance at a known dose. One of the mixtures is analysed immediately and the other is analysed after a given period. The quantity or parameter studied is:
IP_{treated}: the intensity of at least one signal corresponding to the phosphorylated and treated form of one of each type of protein, and/or
IP_{treated}/I_{treated} the ratio between the intensity IP_{treated} and the intensity I_{treated}, I_{treated} being the intensity of the signal corresponding to the marking of each type of treated protein, in its phosphorylated and non-phosphorylated forms, and/or
dIP_{treated}/dt: the variation over time in the intensity IP_{treated}, and/or
d(IP_{treated}/I_{treated})/dt: the variation over time in the ratio IP_{treated}/I_{treated}.

The radiomimetic chemotherapeutic substance causes double-strand breaks in the DNA. It is thus known that a given quantity of radiomimetic chemotherapeutic substance is equivalent to a given level of double-strand breaks and consequently to a given irradiation dose. Thus, in a similar manner to the double sampling of hair follicles, the levels of double-strand breaks at time t=0 and then after a given period Δt are known and it is possible to determine the speed of repair of the double-strand breaks in DNA of each individual. There is then a gain in precision in the measurement of the irradiation dose received. The radiosusceptibility of each individual can be determined by this method. The radiomimetic chemotherapeutic substance is preferably neocarzinostatin (NCS). It will preferably be present in the reaction well at the time when the sample is introduced into this well.

In some embodiments, only one of the two samples is put in contact with a known dose of NCS in order to determine the basic response level of an individual. This is because, in addition to the speed of repair of the DNA, the degree of damage caused to the DNA by the same ionising radiation varies from one individual to another. The quantity or the parameter studied may be:
IP: the intensity of at least one signal corresponding to the phosphorylated form of one type of protein,
IP/I: the ratio between the intensity IP and the intensity I, and/or
IP/IP_{treated}: the ratio between the intensity IP and the intensity IP_{treated}, IP_{treated} being the intensity of at least one signal corresponding to the phosphorylated and treated form of one type of protein, and/or
IP_{treated}/I_{treated} the ratio between the intensity IP_{treated} and the intensity I_{treated}, I_{treated} being the intensity of the signal corresponding to the marking of this type of treated protein, in its phosphorylated and non-phosphorylated forms, and/or
(IP/I)/(IP_{treated}/I_{treated}: the ratio between the intensities IP and I and the ratio between the intensities IP_{treated} and I_{treated}.

In general terms, the quantity used for quantifying the dose received and/or the radiosusceptibility may be a linear or non-linear combination of the aforementioned quantities.

Thus it is possible to carry out mapping for quickly evaluating the local and global irradiation dose of an irradiated individual according to his radiosensitivity.

The irradiation dose or the moment of irradiation can be determined by means of a surface area listing the variation in the degree of phosphorylation of at least one protein according to the irradiation dose received and the delay time of the observation. The irradiation dose or the moment of irradiation can be determined by means of a derivative or a finite increment of said surface area.

The present invention also proposes a device for sampling hairs from an individual, particularly but not exclusively suited to the implementation of the method as described above. It comprises:
a patch fixed to one end of a piston that is able to move in a tube from an advanced position in which the patch extends at least partly outside the tube and can be applied by means of the piston to a sampling region, and a retracted position in which the patch is housed in the tube, the removal of the patch from the region being intended to cause the pulling away of the hairs with their follicles and the retraction of the piston into the tube being intended to cause the introduction of the hair follicles attached to the patch into the tube, and/or
a patch formed by a flexible strip that is fixed by one end to a rod carried by a plug, the strip being intended to be applied to a sampling region and then to be extracted from this region in order to pull off hairs with their follicles in the region, and the strip being configured so as to be coiled around the rod, and/or
a patch comprising a flexible strip of a synthetic plastics material (polymer, etc.) intended both to be applied to a sampling region and then to be extracted from the region in order to pull off hairs with their follicles in the region, and to be applied and stuck to an opening of at least one reaction well with a view to closing it hermetically.

The present invention also proposes a device for sampling the blood of an individual, particularly but not exclusively suitable for implementing the method as described above. It comprises a lancet intended to pierce the skin of the individual. This lancet comprises an ejectable blade and preferably comprises a chamber for storing a solution of alcohol and heparin (or any mixture containing an antiseptic and an anticoagulant), which is intended to be released at the time of ejection of the blade. The chamber is preferentially composed of a cap made from a biological polymer (natural, such as gelatin, a sugar, cellulose, etc.) or artificial polymer disposed in front of the retracted lancet, so that ejecting the lancet causes rupture of the chamber and release of its content.

Finally, the present invention proposes a kit particularly but not exclusively suitable for implementing the method as described above. It comprises at least one device for sampling hairs from an individual, as described previously for example, optionally a device for sampling the blood of an individual, as described previously for example, and at least one multiwell plate. The patches used for sampling the hair are intended to be introduced into different wells or to close different wells of a multiwell plate.

### DESCRIPTION OF THE FIGURES

The invention will be better understood and other details, features and advantages of the invention will emerge from a reading of the following description given by way of non-limitative example and with reference to the accompanying drawings, in which:

FIG. 1 is a flow diagram representing the steps of an embodiment of the method according to the invention,

FIGS. 2 and 3 are schematic views in axial section of a sampling device according to the invention, and depicting respectively two positions of the piston of this device,

FIGS. 4 and 5 are schematic views of another sampling device according to the invention, seen from the side and from above, and depicting respectively two positions of the strip of this device,

FIG. 6 is a multiwell plate in the wells of which the sampling devices are intended to be fitted,

FIG. 7 is a schematic view in axial section of a reaction well in which a device according to FIGS. 2 and 3 has been fitted,

FIG. 8 is a schematic view in axial section of a reaction well in which a device according to FIGS. 4 and 5 has been fitted,

FIG. 9 is a schematic view in axial section of a reaction well containing hairs put in contact with a solution,

FIG. 10 is a schematic view of a blood-sampling device with ejectable blade,

FIG. 11 is a schematic view in axial section of the device of FIG. 10,

FIGS. 12 and 13 are schematic views in axial section of a reaction well and depict steps of recovering and transferring sampled-blood cells,

FIG. 14 depicts schematically the transfer of the content of the reaction wells of a multiwell plate into the reaction wells of another multiwell plate by means of a multichannel pipette,

FIG. 15 is a schematic view in transverse section of a multiwell plate with a removable bottom,

FIG. 16 is a schematic view in transverse section of a multiwell plate with a filtering membrane that is placed on a system for aspirating the content of each well,

FIG. 17 depicts schematically steps of extracting, isolating, filtering and depositing proteins on the bottom of a reaction well, of the method according to the invention,

FIG. 18 depicts schematically steps of extracting, isolating, filtering and capturing proteins by means of antibodies fixed on the bottom of a reaction well, of the method according to the invention,

FIG. 19 depicts schematically various steps of marking proteins in the reaction wells,

FIG. 20 depicts schematically the bottom of a reaction well, comprising several regions for fixing specific antibodies,

FIG. 21 is a schematic view of an LIBS analysis apparatus,

FIG. 22 is a spectrum showing the results of an LIBS analysis of sampled proteins,

FIG. 23 is a graph showing the change in the intensity of an LIBS signal as a function of the irradiation dose received by an individual,

FIG. 24 is a comparative curve between the FACS and LIBS detection methods,

FIG. 25 depicts the change in the direct intensity of fluorescence as a function of the irradiation dose received by an individual,

FIG. 26 is a graph showing the change in the fluorescence intensity as a function of the irradiation dose received by an individual,

FIG. 27 is a graph showing the change in the amount of apoptotic cells in a mixture as a function of the quantity of NCS in this mixture,

FIG. 28 is a graph showing the change in the number of apoptotic cells as a function of the irradiation dose received by an individual,

FIG. 29 shows the change in the quantity of Ku70 proteins in cells exposed to a single irradiation of 10 Gy to the left on the drawing, and to a plurality of successive irradiations of 1 Gy, once per week for seven weeks, to the right on the drawing (NT: non-treated proteins),

FIG. 30 shows the quantity of Ku70 proteins in cells exposed to an irradiation of 10 Gy, 30 minutes and 24 hours after the irradiation (NT: non-treated proteins),

FIG. 31 shows the difference in performance between fluorescence measurements on H2AX proteins of cells irradiated at 0, 2 and 8 Gy carried out by an antibody (Millipore) and by a Fab-zipper antibody,

FIG. 32 shows the locations of patches on an individual, and

FIGS. 33a, b and c are graphs showing the change in the fluorescence signal of an anti-Ku70 marking in FACS for extracts of proteins of human cells (ZR75.1) irradiated at 10 Gy (grey) or not (black), and captured on particles of hydrogenated nanodiamonds, with various extraction modes,

FIG. 34 shows an example of perfecting the extraction of proteins by means of microwaves,

FIG. 35 shows the implementation of a transfer of the dot blot type of samples irradiated at 0.5 Gy on membranes, and

FIGS. 36 to 41 are graphs illustrating an example of a quantification method according to the invention.

### DETAILED DESCRIPTION

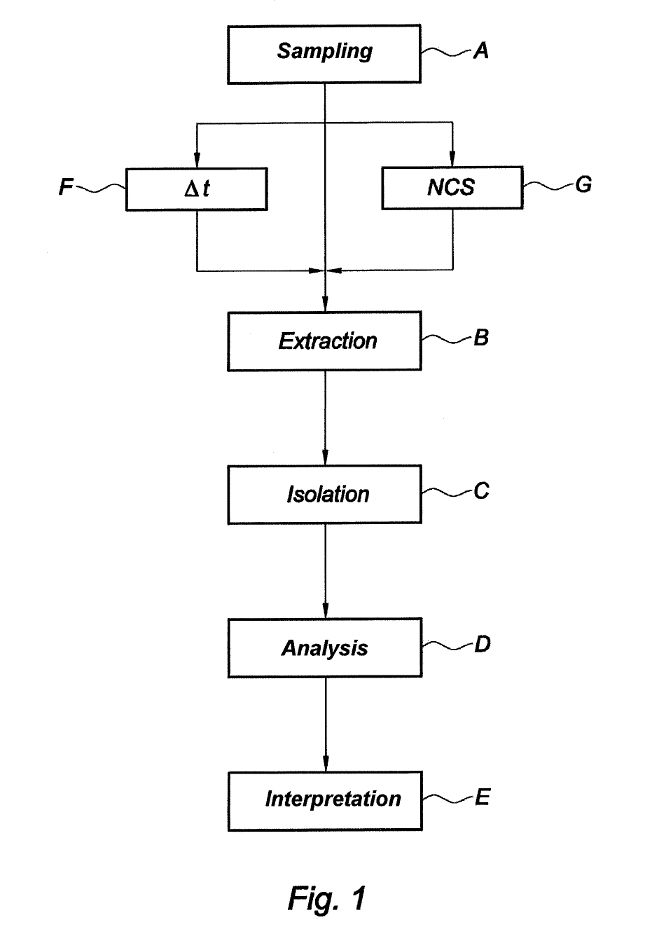

Reference is made first of all to FIG. 1, which is a flow diagram representing the various steps of an embodiment of the method according to the invention for evaluating by biodosimetry the irradiation dose received by an individual who has been subjected to ionising radiation.

In this embodiment, the method comprises essential steps and optional steps. The essential steps are a step A of sampling biological materials from at least one region of the body of the individual, a step of extracting B and isolating C the proteins contained in these biological materials, and a step of analysing D and interpreting E the analysis results in order to determine the irradiation dose received in the or each sampling region. The optional steps F and G are described in detail below.

The sampling step A comprises the sampling, in at least one region of the body of the individual, of bristles and/or hairs and/or blood. The bristles and hairs comprise hair follicles or bulbs, the cells of which contain proteins. The blood comprises red corpuscles and cells of interest that also contain proteins. Analysis of the proteins contained in the hair follicles makes it possible to locally evaluate the irradiation dose received by an individual whereas analysis of the proteins contained in the blood cells makes it possible to globally evaluate the irradiation dose received by the individual.

As explained above, one of the major consequences of ionising radiations for cells is double-strand breaks (DSBs) in DNA. The proportion of DNA double-strand breaks in an individual is directly proportional to the irradiation dose received by this individual. Measuring these breaks therefore makes it possible to determine the irradiation dose received by the individual.

DNA double-strand breaks cause a series of cell responses, including the activation of protein and enzyme systems for signalling and repairing damage to the DNA. This response in particular involves the proteins of the ATM system, also referred to as ATM cascade. Some proteins in the ATM system such as H2AX, Chk2, NBS1, ATM, BRCA1, 53BP1, P53, KU70, etc. are phosphorylated whereas others are dephosphorylated. One of the proteins phosphorylated early in response to double-strand breaks is the histone H2AX. This histone is phosphorylated into γ-H2AX.

In the case where an individual has been irradiated in a region of the body, the step of analysing the cells sampled in this region will reveal the presence of at least one type of phosphorylated protein of the ATM system, quantifying which will make it possible to evaluate the irradiation dose received in this region. One type of protein in the ATM system comprises H2AX, Chk2, NBS1, ATM, BRCA1, 53BP1, P53 or KU70. Each type of protein comprises the phosphorylated and non-phosphorylated forms of this type (for example H2AX and γ-H2AX). In a variant, analysing the cells sampled in each region will reveal the presence of at least one acetylated protein type.

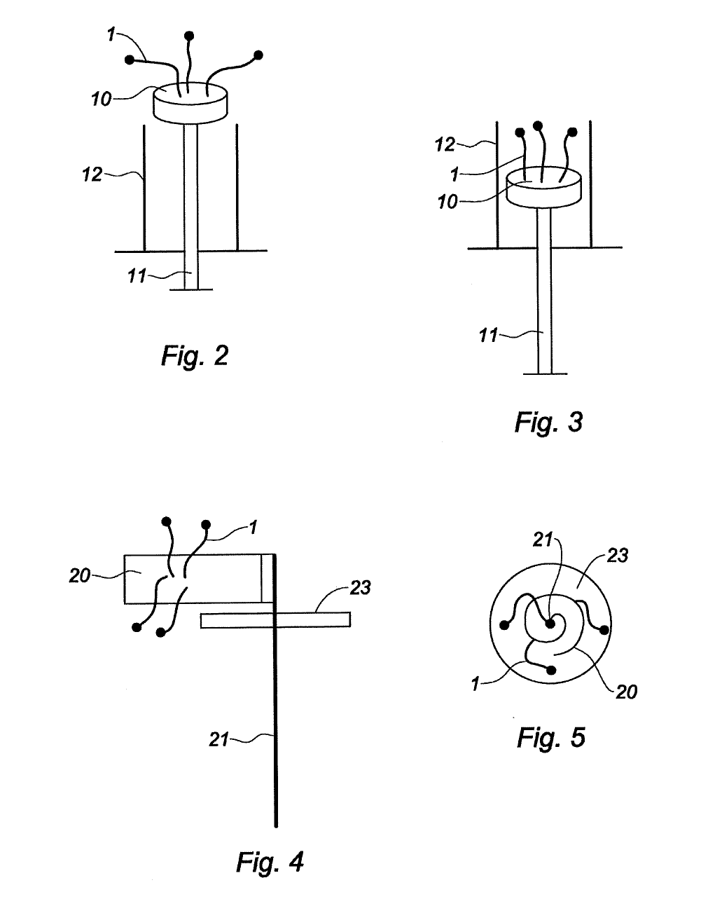

FIGS. 2 to 5 show two embodiments of a hair sampling device. In these two embodiments, the sampling is done by applying a patch to the skin of the individual by means of a special applicator which makes it possible to fold the hair follicles after pulling off the hairs for the purpose of being able to introduce them easily into a reaction well. The patch may be composed of wax, adhesive, glue or any other sticky material making it possible to perform a depilation.

In the embodiment in FIGS. 2 and 3, the patch 10 has a thin pellet shape with a substantially cylindrical contour and is fixed to one end of a piston 11. This piston is able to move in translation inside a tube 12, from an advanced position where the patch 10 is outside the tube and can be applied to the skin (FIG. 2) and a retracted position in which the patch is housed in the tube (FIG. 3).

After application of the patch 10 (in its advanced position) to the sampling region by means of the piston 11, removing the patch 10 causes the hairs 1 to be pulled off (FIG. 2). Movement of the piston 11 in the tube 12 (FIG. 3) to its retracted position causes the folding of the hairs 1, which thus easily enter the tube 12.

As shown in FIG. 7, the open end of the tube 12, by means of which the patch 10 can emerge, is configured so as to be able to be fitted in a reaction well 3. The hairs that are housed in the tube 12 are thus easily introduced into the reaction well 3. Once fitted in the reaction well 3, the function of the sampling device is to sealingly close the reaction well.

In the embodiment in FIGS. 4 and 5, the patch 20 is in the form of a substantially rectangular flexible strip, one end of which is fixed to one end of a rod 21 so that the strip extends substantially in a plane passing through the longitudinal axis of the rod 21.

The rod 21 carries, in the vicinity the patch 20, an external annular collar forming a plug 23. After the patch 20 is applied to the skin, removing it causes the hairs 1 to be pulled out (FIG. 4). The patch 20 is then coiled around the rod 21, that is to say around the longitudinal axis of the rod. In its coiled position shown in FIG. 5, the patch 20 does not project beyond the external periphery of the plug 23. The patch can be held in this position by means of an adhesive (for example a double-face adhesive). The patch 20 can thus easily be introduced into a reaction well 3, as shown in FIG. 8, the plug 23 being intended to hermetically close the reaction well 3.

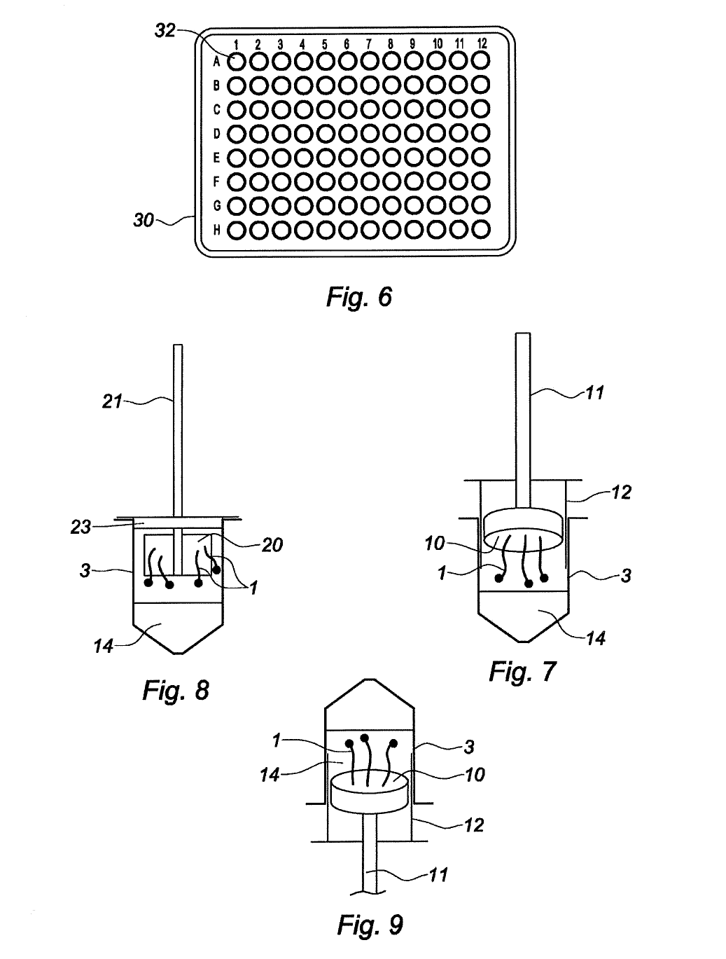

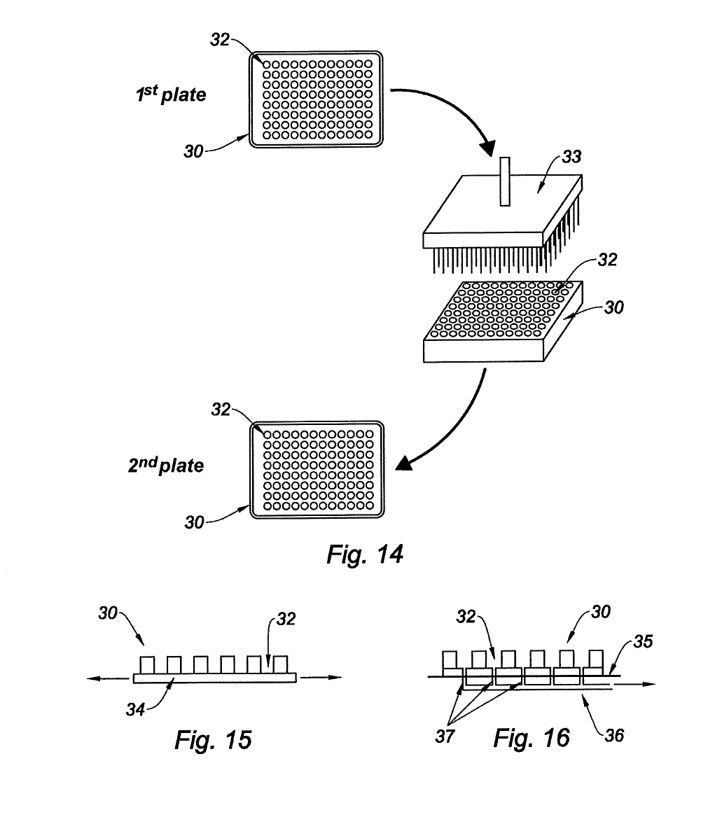

Preferentially, the reaction well 3 is the well 32 of a multiwell plate 30 (FIG. 6). Each well 32 preferably comprises the hairs from a given sampling at a given instant and on a given region of a given individual. The use of a multiwell plate 30 has in particular the advantage of being able to sample, add and/or transfer the content of the wells 32 on a single occasion by means of a multichannel pipette 33, for example in an automated manner (FIG. 14).

After the patch 10 is introduced into the reaction well, the hairs 1 are put in contact with either a non-denaturing solution or cell lysis solution 14. This step can be implemented for example by the following successive substeps: adding the solution 14 to the reaction well 3, introducing the patch into the reaction well until sealed closure of the well is obtained (FIG. 7 or 8), and then turning over the reaction well or the multiwell plate 30 so that the hairs are put in contact with the solution 14 (FIG. 9).

According to a protocol for extracting proteins from sampled hairs, the equipment used comprises a microwave, a 96-well plate (Eppendorf®) with lid, and a wax strip (for example Nair®). Wax strips measuring 1 cm by 3 cm are cut. The sampling region is depilated by means of a strip. A 50 μl solution of 1×TBS is introduced at the bottom of three reaction wells in the plate. The strip is stuck to the openings of the wells so that the hairs are situated in the wells and these wells are closed hermetically by the strip. The plate is closed with its lid and is then turned over while tapping thereon in order to make the solution fall onto the hairs and the strip. The plate is microwaved for 15 seconds at 450 W. The plate is turned over while tapping thereon in order to make the solution fall to the bottom of the reaction wells. The lid and the wax strip are removed. The content of the reaction wells is transferred into the wells of another plate for dot blot study or the like.

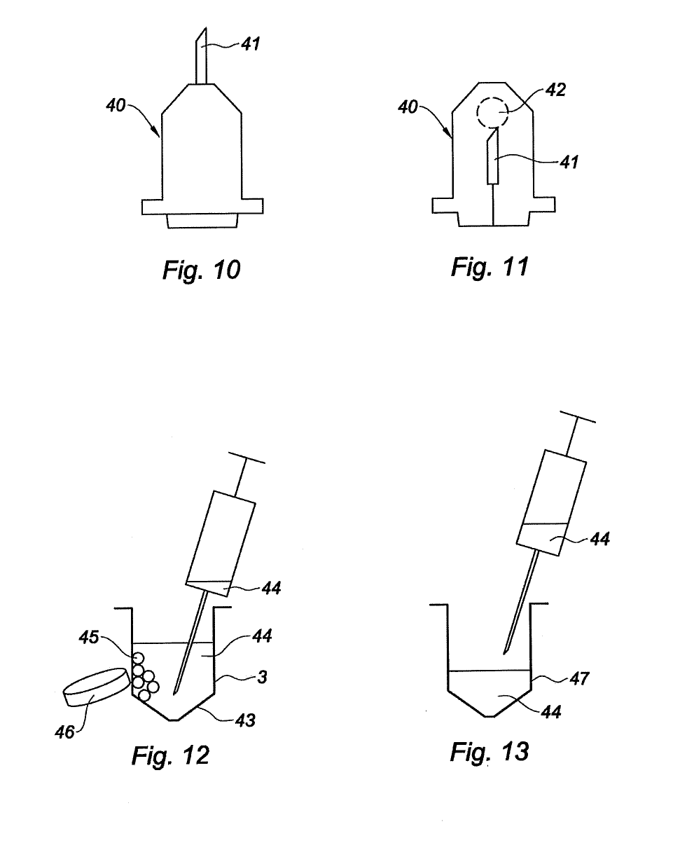

FIGS. 10 and 11 show an embodiment of a blood-sampling device, which comprises here a lancet 40 comprising an ejectable blade 41 that will make it possible to prick and pierce the skin, for example of the finger, of an individual. It may be a disposable lancet similar to those intended for blood sampling in diabetics.

Before any blood sampling, it is necessary to disinfect the sampling region by means of an antiseptic solution. Preferably, this will be alcohol. For the purpose of facilitating the sampling and harvesting a sufficient quantity of blood to carry out the test, it is possible also to apply to the region an anticoagulant solution, such as heparin. These two solutions may be applied using any application means, such as a sterile compress.

It is also possible to store the antiseptic/anticoagulant mixture in a storage chamber 42 that is housed inside the lancet on the ejection path of the blade 41, and which will be pierced when the blade 41 is ejected so that the mixture is spread over the sampling region simultaneously with the piercing of the skin (FIG. 11).

To carry out all the biodosimetry tests, 120 to 150 μl of blood can be collected per individual in a reaction well 43, such as a graduated tube (FIG. 12).

To prevent any haemorrhage, this sampling step may be followed by an additional step consisting of applying a solution of coagulant, such as calcium chloride (CaCl₂), on the sampling region. This step may be very useful in the case of sampling on a haemophilic individual for example. A compress containing a solution of fibronectin may be applied after the application of the calcium chloride solution.

The dosimetry is done on blood cells, with the exception of the red corpuscles, and the red corpuscles are therefore separated from these cells in order to facilitate analysis. For this purpose, the reaction wells 43 previously contain a suspension of magnetic balls 45 functionalised with antibodies against blood group antigens (such as anti-A, anti-B, anti-rhesus+, anti-CD235, etc.) and optionally an anticoagulant in order to prevent natural coagulation of the blood. The functionalised magnetic balls will cause the formation of a red-corpuscle clot while the other cells will remain in the residual liquid 44. The clot 45 is formed in approximately 1 to 4 minutes. When the residual liquid 44 is taken off, the clot and the magnetic balls are then separated from the residual liquid by means of a magnet 46 (FIG. 12).

50 μl of residual liquid can be introduced into a second reaction well 47 (FIG. 13), which is then hermetically closed. The second reaction well may be a well 32 in a multiwell plate 30 (FIG. 6). Each well preferably comprises the residual liquid taken from a given individual at a given instant.

The blood sampling is preferentially carried out before the sampling of the hairs. Thus the sampling of the hairs can be carried out, for example, during the time waiting for the formation of clots caused by the suspension of magnetic balls functionalised with antibodies.

According to a particular blood sampling protocol, the equipment used comprises mouse anti-IgM magnetic balls (Ademtech), mouse anti-blood group (A, B, CD235a, etc.) antibodies (IgM), a magnet, and the sampled blood. 20 μl of anti-IgM balls and 15 μl of anti-blood group antibodies are added in a 500 μl tube (for example Eppendorf®). A drop of blood (between 20 and 50 μl) is added to the tube. After waiting for approximately 15 seconds (possible presence of a vortex), the tube is placed against the magnet for approximately one minute. Once the balls have taken away the maximum amount of erythrocyte, the blood plasma containing the leucocytes and thrombocytes can be taken off and studied.

The step following the sampling A is the step B of extracting the proteins from the cells contained in the biological material sampled. This step consists either (i) of putting the cells in contact with a cell lysis solution, or (ii) putting the cells in contact with a non-denaturising solution and then subjecting the cells to a mechanical lysis or one by means of waves.

In the first case (i), in a preferential embodiment, a cell lysis solution 14 is present in each reaction well 3. In the case of the sampling of hairs, these may be put in contact with this solution, for example by turning over the reaction well, as shown in FIG. 9. In the case where the reaction well belongs to a plate 30, this plate may be turned over in order to simultaneously turn over all the reaction wells.

In the case of blood sampling, the residual liquid 44 containing the blood cells of interest can be put in contact with the cell lysis solution when the residual liquid is transferred into a reaction well 47, as shown in FIGS. 12 and 13.

Putting the blood cells or hair follicles in contact with the cell lysis solution causes the destruction of the cells and the release of the proteins contained in these cells. The protein extraction may last for between 15 seconds and 3 minutes approximately.

In the second case (ii), the reaction wells may contain a non-denaturing solution such as TBS, TBE, etc., preferably free from phosphorus in order not to interfere with the analyses by LIBS. The lysis may be caused in several different ways and preferably by microwaves, so as not to modify the state of phosphorylation of the proteins. The multiwell plate may, after being turned over, be placed under a microwave source for between 1 and 60 seconds so as to burst the cells and release the proteins that they contain.

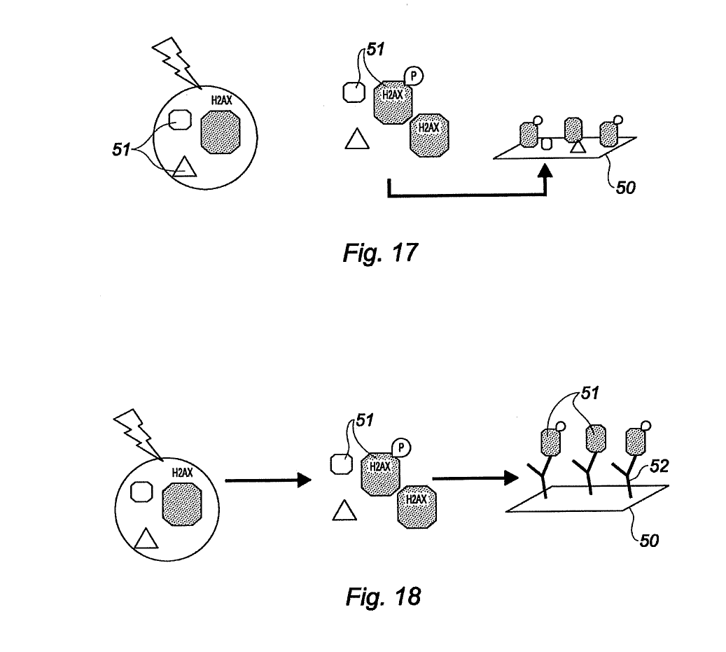

After extraction B of the proteins 51, they are captured and isolated C. They may be captured:
either directly on the bottoms 50 of the reaction wells, so as to form non-structured systems (FIG. 17),
or by means of antibodies 52, so as to form structured systems after transfer into a well plate where the bottom of the wells comprises a structured antibody system where for example each type of antibody is printed in the structure of a pattern, for example by means of an inkjet printer (FIG. 18).

In each non-structured system (FIG. 17), all the types of protein are absorbed on the bottom 50 of a reaction well, in their phosphorylated and non-phosphorylated forms. In the example shown in FIG. 17, the elements with a substantially octagonal shape represent one type of protein, the histones H2AX. When these proteins are phosphorylated (γ-H2AX), they carry a marked element P. In the bottom of the reaction well in FIG. 16, three proteins of the H2AX type are present, including two phosphorylated (γ-H2AX) and one non-phosphorylated (H2AX).

In each structured system (FIG. 18), the bottom 50 of each well is structured with antibodies 52 grafted onto this bottom. These antibodies 52 are directed against a single type of protein. In the example shown, the antibodies are intended to bind specifically to the H2AX proteins, in their phosphorylated and non-phosphorylated forms. In the bottom of the reaction well in FIG. 18, three proteins of the H2AX type are present, including two phosphorylated (γ-H2AX) and one non-phosphorylated (H2AX). The other types of protein are not retained on the bottom of the well and can be eliminated.

Different antibodies intended to bind specifically to various types of protein can be grafted at the bottom of each reaction well, as is shown schematically in FIG. 20. The bottom 50 of the well comprises a plurality of separate regions X for fixing antibodies 52, the antibodies in one region being different from the antibodies in the other regions and being intended to bind to another type of protein different from the types of protein intended to be fixed to the antibodies in the other regions. The regions X may comprise for example antibodies corresponding respectively to H2AX, Chk2, NBS1, ATM, BRCA1, 53BP1, P53 and KU70, in their phosphorylated and non-phosphorylated forms.

At the end of the isolation step, the proteins to be analysed are therefore retained on the bottoms of the reaction wells, according to a particular embodiment of the method according to the invention. Preferentially, the multiwell plate 30 will comprise a removable bottom 34 that facilitates the reading of the results during analysis (FIG. 15).

The bottom may also comprise or be formed by a filtering membrane 35 through which the solutions contained in the wells 32, and optionally certain molecules of a given size, can be aspirated (FIG. 16) the aspiration may be done via a support plate 36 intended to receive the multiwell plate 30 and comprising aspiration capillaries 37 that each comprise a first end emerging opposite a well 32 and a second end connected to an aspiration source (FIG. 16). Such a system makes it possible to empty all the reaction wells 32 very quickly in order to accelerate and facilitate for example the fixing of the proteins on the bottom of the wells. The bottom may also comprise or be formed by a membrane capable of absorbing the proteins. It may be a membrane made from PVDF (standing for polyvinylidine difluoride), nitrocellulose or epoxy.

After isolation C of the proteins 51, various solutions exist for their analysis D.

In a preferred embodiment of the method, the analysis may be effected by measuring the intensity (IP) of a marker particular to the phosphorylated form of a type of protein in question, or by the ratio of this intensity (IP) to the intensity (I) of a marker of the phosphorylated and non-phosphorylated forms of this type of protein (this ratio IP/I corresponding to the degree of phosphorylisation of this type of protein). By comparison with pre-established calibration curves, it is possible to determine the irradiation dose received by an individual.

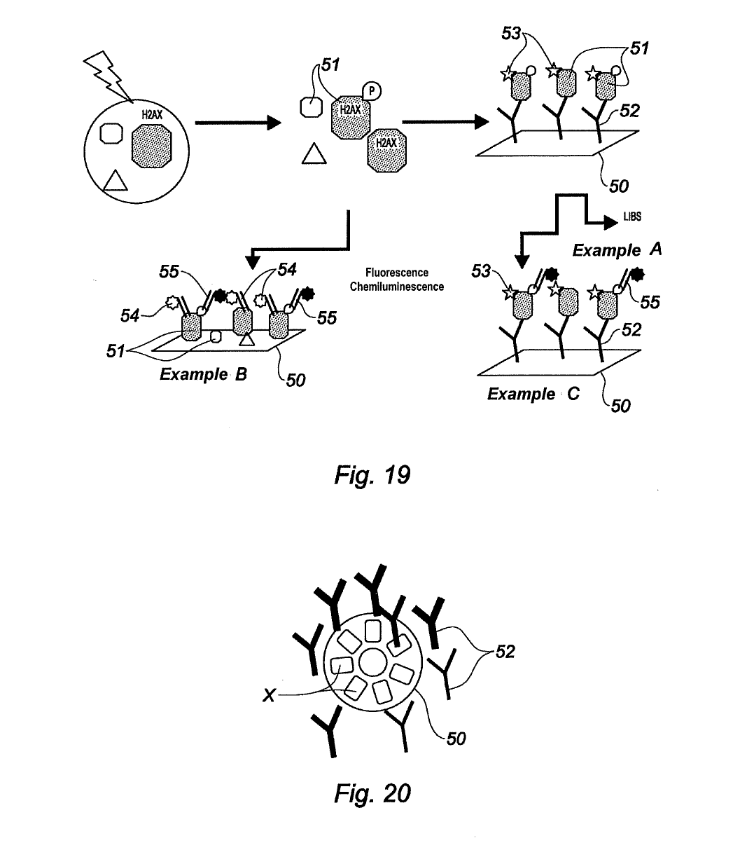

FIG. 19 shows schematically three examples A, B and C of marking of the proteins at the bottom of the reaction wells.

According to example A, all the proteins (phosphorylated and non-phosphorylated forms) of the type of protein retained on the bottom of the reaction well, as explained with reference to FIG. 18, are marked with boron 53 with a view to an LIBS analysis.

According to example B, as explained above with reference to FIG. 17, all the proteins 51 contained in a reaction well are deposited on the bottom of this well. The phosphorylated and non-phosphorylated forms of a type of protein are marked by means of a first marker 54, and the phosphorylated form of this type of protein is marked by means of a second marker 55.

Example C is similar to example A but also comprises markers 56 particular to the phosphorylated form of the type of protein in question.

In examples B and C, the marking of the proteins can be done by means of antibodies, these antibodies being able to be chimeric molecules as described in the prior application PCT/FR2011/050812 of the applicant.

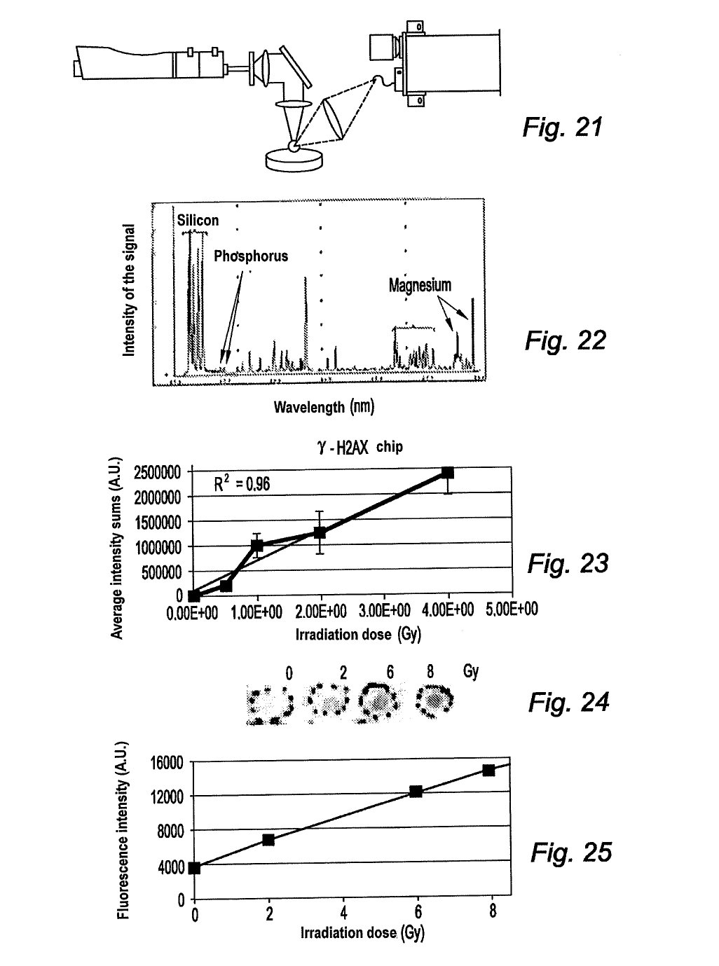

In example A, the bottom 50 of the reaction well or of the multiwell plate can be analysed by a method able to directly measure the level of phosphorus in the phosphorylated proteins. It may be an analysis method with a laser of the LIBS type (FIG. 21). This method makes it possible to obtain a spectrum (FIG. 22) for determining the intensity (IP) of the signals corresponding to the phosphorus, which is proportional to the quantity of phosphorylated proteins, as well as the intensity (I) of the signals corresponding to the boron (marker 53), which is proportional to the quantity of phosphorylated and non-phosphorylated proteins. By comparison with calibration curves, such as the one shown in FIG. 23, it is possible to determine the irradiation dose received by an individual.

The calibration curve in FIG. 23 represents the change in the intensity IP as a function of the irradiation dose received by an individual. This curve was traced from four points corresponding respectively to four tests carried out on four different individuals. Hairs from each individual were sampled and subjected to ionising radiations at a predetermined dose. Their proteins were extracted and isolated, as described above, and then analysed by the LIBS method. The results obtained made it possible to establish the curve in FIG. 23, which makes it possible to determine the irradiation dose received by an individual from the intensity IP. A calibration curve of the type in FIG. 23 is particular to a given type of protein and to a given parameter or quantity studied. In the aforementioned example, the parameter observed is IP. In a variant, the parameter studied is the ratio IP/I. The calibration curve is then of the type IP/I=f(D), D being the irradiation dose.

It was checked that the results obtained by the LIBS analysis step are close to those obtained with the so-called FACS cell sorting method of the prior art, which makes it possible to validate the results.

In the aforementioned examples B and C, the marked proteins may be analysed by fluorescence, chemiluminescence or colorimetry, the markers 53, 54, 55 being fluorescent, colorimetric or chemiluminescent markers.

FIG. 24 shows the intensity of fluorescence of γ-H2AX proteins, captured directly on the bottom of four reaction wells provided with a PVDF membrane, these proteins having been subjected to different irradiation doses (0, 2, 6 and 8 Gy). The increase in the intensity IP of fluorescence as a function of the increase in the irradiation dose can be observed and a calibration curve can be established (IP=f(D)—FIG. 25). This curve makes it possible to determine the irradiation dose received by an individual as a function of the fluorescence intensity measured, particular to the phosphorylated form of a type of protein for example. As indicated above, the calibration curve may be of the IP/I=f(D) type.

In a variant embodiment of the method according to the invention, after extraction, separation and marking of the proteins, as described above, the marked proteins are transferred into the wells of a multiwell plate, so that each well comprises a mixture of particles on which antibodies against given markers are fixed, for example markers of the non-phosphorylated form of a type of protein.

These particles preferably have micrometric sizes, for example between 10 nm and 100 mm. These may be a mixture of particles such that each particle comprises a single type of antibody.

In this configuration, a single particle size may be attributed to a type of antibody so as to be able to distinguish the particles by their size and to be able to deduce therefrom the antibodies that are fixed thereto. In other cases, each particle may comprise a mixture of antibodies.

Once the markers have been captured by the antibodies of the particles, various analyses may be carried out:
the particles may be trapped by filtration on the membrane constituting the bottom of the multiwell plate, as explained above, the membrane constituting a filter, the pores of which have a size less than the size of the particles,
a cocktail of antibodies comprising antibodies directed against epitopes other than the epitopes recognised by the antibodies fixed to the particles, and such that each type of antibody comprises a given fluorescent marker, may be present or added with the particles. Each marker fixed to a particle will then be marked by one or two different additional antibodies; preferentially, by an antibody against a phosphorylated form of the marker being researched.

After filtration, the particles retained on the bottom of the multiwell plate comprise the markers trapped on the particles, the markers being marked firstly in a ubiquitous fashion or by an antibody, secondly by the phosphorylated markers, and optionally in addition marked by an antibody against the phosphorylated form. The bottom of the plate is then analysed by one of the aforementioned methods.

Another alternative is the analysis of the particles in each well in the plate by FACS.

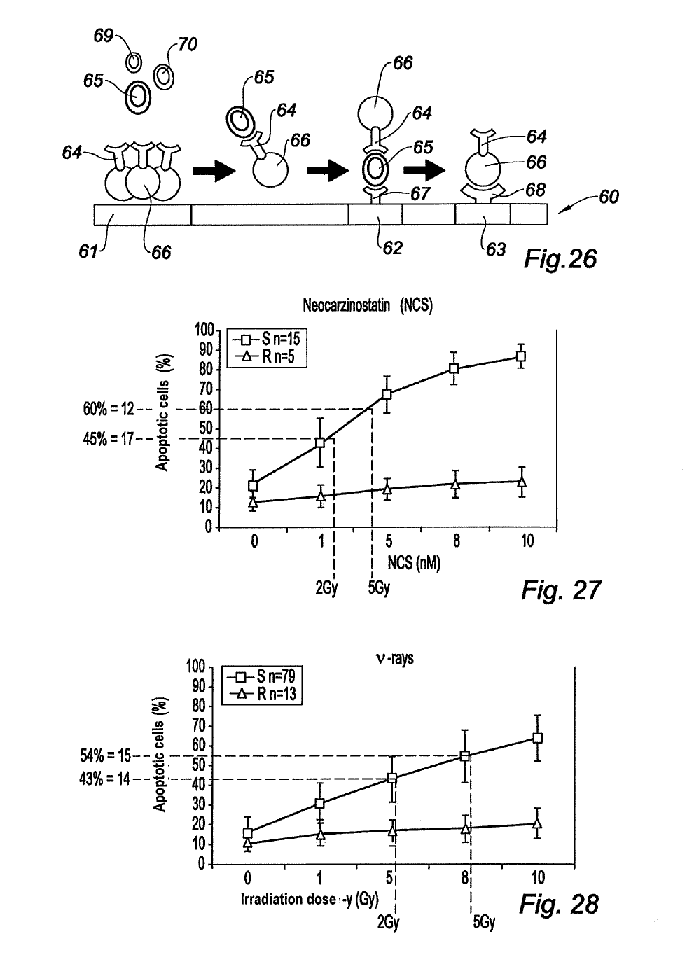

In another variant embodiment of the method according to the invention depicted in FIG. 26, after extraction, the proteins are transferred into the wells of a multiwell plate so that the bottom of each well is covered by a strip 60 of Whatman® paper, this strip having an elongate shape and defining at one end a region 61 of deposition of the proteins to be analysed, and defining at the opposite end analysis regions 62, 63. The proteins are intended to migrate from the deposition region to the chromatography analysis region.

The deposition region 61 comprises antibodies 64 against the phosphorylated form 65 of a type of protein (here γ-H2AX), these antibodies 64 being fixed to particles 66, such as gold balls.

The first analysis region 62 comprises fixed antibodies 67 against the phosphorylated and non-phosphorylated forms of the type of protein (here H2AX). The second analysis region 63 comprises fixed antibodies 68 intended to capture the particles 66, the antibodies 64 of which are not bound to proteins (since the particles 66, the antibodies 64 of which are bound to proteins 65 are retained in the region 62).

A solution containing the proteins to be analysed is deposited in the region 61 of the strip 60, this solution comprising the type of protein studied in its phosphorylated 65 (γ-H2AX) and non-phosphorylated 69 (H2AX) forms. This solution may be obtained by extraction from the blood or hair proteins as described above. A predetermined quantity of marked proteins 70 of the aforementioned type, in their non-phosphorylated form, is added to the solution before deposition on the strip.

When this known quantity of marked proteins 70 is deposited alone and analysed with the strip 70, the results obtained, for example by colorimetry, supply a signal in the region 62, the intensity i* of which is maximum (100%) since all the marked proteins are retained in this region.

During a test, the intensity i of the signal obtained in the region 62 will be all the more diluted, the greater the number of proteins of the aforementioned type (in its phosphorylated and non-phosphorylated forms) retained in this region. The values of i* and i make it possible to determine the parameter I, that is to say the quantity of proteins of the aforementioned type, extracted from the biological material sampled on the individual, by the formula: I=(i*−i). The parameter IP is obtained by the intensity of the signal generated by the balls 65 in the region 62. The parameters i/i* (that is to say Iₛₐₘₚₗₑ/I_{ref}) where i*/I (I_{ref}/I) can also be studied.

As before, by comparing with a calibration curve, it is possible to determine the irradiation dose received by an individual.

The modification of the degree of phosphorylisation of the proteins involved varies in particular according to the speed of repair of the DNA double-strand breaks. This speed of repair is different from one individual to another (interindividual variations). It is therefore advantageous to measure this speed in order to correct the measurement and obtain much more precise results (step F in FIG. 1).

This speed may be determined by means of two blood or hair samples in the same region. These samplings are carried out at a predetermined interval of time Δt or are carried out almost simultaneously, and at least one of the samples is cultured for a predetermined period Δt. The period Δt may be between 10 minutes and 6 hours.

Each sample is treated and analysed as described previously. The measurements of intensity IP or of ratio IP/I obtained may be accompanied by the derivative or finite increment of this value. This thus gives access to the couple of parameters (IP/I, ∂(IP/I)/∂t) or (IP, ∂IP/∂t). The parameters ∂(IP/I)/∂t and ∂IP/∂t can thus be studied and the calibration curves can be of the type ∂IP/∂t=f(D) or ∂(IP/I)/∂t=f(D).

It is also possible to correct the errors due to interindividual variation using a radiomimetic chemotherapy substance. This is a chemical irradiation mimic, i.e. a substance which, when it is added to biological material, causes DNA double-strand breaks (step G in FIG. 1).

The substance used is for example neocarzinostatin (NCS). The graph in FIG. 7 shows the change in the ratio of apoptotic cells of a mixture as a function of the NCS concentration in this mixture, for two different cell lines, respectively sensitive S and resistant R. The graph in FIG. 28 shows the change in the ratio of apoptic cells in a mixture as a function of the irradiation dose received by this mixture, for the aforementioned two lines. These graphs made it possible to determine in particular that a concentration of 1 nM of neocarzinostatin is equivalent to, that is to say produces the same effect as, an irradiation dose of 1.2 Gy. Several hair samplings are carried out on the same region of an individual, along with a plurality of blood samplings. Two of the samples are subjected to the action of NCS at a known dose, for example equivalent to 1 Gy, in reaction wells in a plate. Each sample can be treated and analysed, as described previously. One of the two samples is analysed immediately at the same time as a sample that has not received treatment, and the second is analysed after a given period Δt, for example 1 hour.

The measurements of intensity IP or of ratio IP/I obtained may be accompanied, in addition to derivatives of these values, by parameters taking account of the interindividual variations of individuals. The measurements IP/I obtained are then modulated by ∂(IP/I)_{treated}/∂t and by (IP/I)/(IP_{treated}/I_{treated}).

Sampling curves and/or surfaces can thus be produced by linear or non-linear combination of all or some of these quantities and their ratio (for example the X-axis is the time t, Y is a parameter studied such as IP, and Z is the irradiation dose). All the values and/or linear and non-linear combination of quantities coming from the phosphorylisation measurements may be studied parameters.

In general, hereinafter, the value adopted for measuring the degree of phosphorylation will be referred to as the observable.

In some embodiments, in order to refine the correlation of the measurement of the observable with the dose received, the method takes into account the blood formula of the individuals. The blood formula of the individuals is therefore produced systematically at the time of the blood sampling so that, for an individual i, the blood fractions of the various cell types j making up his blood are defined by Xⱼ. Many portable automated devices make it possible to determine, in a few minutes, the blood formula of an individual, the objective being to determine which fraction of the blood formula it is necessary to consider in order best to correlate the phosphorylation measurements with the dose received.

Thus a method according to the invention comprises a step of seeking a linear combination of the blood formula as a function of the observable and maximising the correlation with the dose received.

The standard curves of surfaces for carrying out this dosimetry will be produced from a reference population.

Preferentially, the reference population will comprise P persons, P being equal to or greater than N, which represents the number of fractions enumerated in the blood formula in question.

The intensities IP or the ratios IP/I, or more generally the observable, are determined on the blood cells of the blood samples from the individuals, as explained above. The observable is measured for various doses varying for example from 0 to 8 gray, in steps of 0.1 gray, and for various times after the exposure varying for example from 0 to 12 hours, in steps of 30 minutes.

For each time t and each dose D_{d} received by an individual i, the blood formula is produced and the equation:

(Σⱼ₌₁^{N}A_{dtij}X_{dtij})(observable_{dti})+f_{dti}=D_{d}

is established where X_{dtij} represents the cell fraction j for an individual i, A_{dtij} the weighting coefficient of the fraction X_{dtij}, making it possible to link the observable_{dtij} to the dose D_{d}; N represents the number of fractions considered in the blood formula, and f_{dti} the correction factor for the correlation.

In order to estimate the factors A to be taken into account for each dose and at each time, all the combinations C of N persons in P are considered.

For each combination C of N persons and for each time t and each dose D_{d}, the system [(Σⱼ₌₁^{N}A_{dtij}X_{dtij}) (observable_{dti})+f_{dti}=D_{d}] of N equations with N unknowns is resolved.

For each system of equations of a combination C, N sets of N factors A_{dtj} and N correction factors f_{dti} are obtained. For each time and each dose, and for each fraction J, a mean weighting factor value A_{dtj} is then calculated from all the A_{dtij} of all the combinations. For each time and each dose the correction factor f_{dt} is calculated from all the f_{dti} obtained for all combinations.

For all doses of the same time, a combination of the A_{dtj} is obtained where J is between 1 and N, such that the correlation: A_{dtj}(observable_{dt}) versus Dose_{d} is the best.

In summary, for each time, a linear combination of the A_{dtj} is sought making it possible to correlate best the dose/effect relationship observed.

In a preferred embodiment, the protein Ku70 is used as a marker for determining the irradiation dose received by an individual.

This is because the degree of phosphorylation of the protein Ku70 is proportional to the dose received. More particularly, quantification of the phosphorylation at the serine 27 of the protein Ku70 makes it possible to estimate the irradiation dose received by an individual.

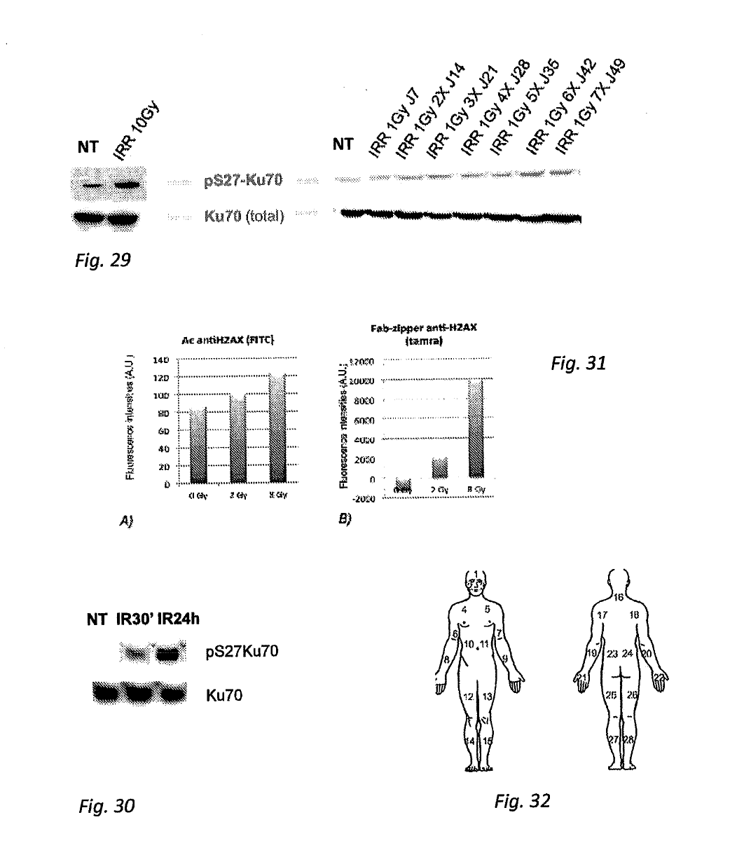

FIG. 29 shows that phospho-Ser27-Ku70 can be induced in the cell lines (T lymphoblasts) exposed to gamma radiation (ionising irradiation) at high single doses (10 Gy) as well as in cells exposed to doses divided up at the rate of 1 Gy/week for 7 weeks (cumulative total dose 7 Gy). Observing the variation in Ku70 makes it possible to quantify chronic irradiation.

FIG. 30 shows that the degree of phosphorylation of the protein Ku70 varies over time. The degree of phosphorylation, 24 hours after irradiation, is appreciably greater than that 30 minutes after irradiation (10 Gy).

In some embodiments, quantification of the phosphorylation will be done by means of an antibody directed against an epitope comprising serine 27. More particularly, the antibody will be directed against all or part of the peptide sequence ENLEA(p)SGDYK (SEQ ID NO:1) (this is here the conventional nomenclature of amino acids, the letter p however representing a phosphate).

In some embodiments, the measurement used for quantifying the irradiation dose of an individual is the ratio of the level of phosphorylated Ku70 (Ku70 p) with respect to the total Ku70 (as presented in FIGS. 29 and 30).

The total quantity of Ku70 remains constant in the cells, and the ratio Ku70 p/total Ku70 makes it possible to determine the irradiation dose received. In some embodiments, the analysis of total Ku70 dose can be done by means of an antibody directed against the parts of Ku70 preserved and not modified, in particular not modified by the phosphorylation which, preferably, is remote from the phosphorylation site, for example by more than 10 amino acids.

The quantification of total Ku70 dose also makes it possible to standardise the other markers used for analysing the irradiation. In particular the ratio γH2AX/total Ku70 makes it possible to evaluate the dose received by an individual even six hours after irradiation. This is because it is shown on FACS that the level of phosphorylation of the protein H2AX varies over time because of the repair of the DNA, and that this variation differs according to the individual. The ratio γH2AX/Ku70 makes it possible to classify the doses received by various individuals in at least three irradiation categories, for example [0-0.5 Gy][0.5-3 Gy][>3 Gy], up to six hours after irradiation.

In some embodiments:
a. the proteins in the cell extract are captured on a membrane substrate, glass, Kapton® or any other material, through a deposition, filtration or Cytospin method. In other embodiments, the proteins are fixed on particles and more particularly on particles of diamond, polystyrene or any other material. The fixing of the proteins on the substrate may be covalent, dative (van der Waals) or simply absorbed,
b. the proteins fixed on the membrane or particles are put in contact with the antibodies directed against the targets such as phosphor Ku70 or γ-H2AX and also against at least one target used for standardisation such as Ku70, H2AX or any other protein expressed consistently in the cells. An antibody directed against a target will have a specific fluorescent or colorimetric marking,
c. after elimination of the non-complexed marked antibodies, reading the fluorescence, where the intensity of each colour corresponding to a given antibody makes it possible to determine the abundance of the target and therefore the degree of phosphorylation, in particular by standardising the measurement by the intensity of fluorescence of at least one standardisation antibody such as I AC_{target}/I AC_{standardisation}, makes it possible to determine the proportion of phosphorylated proteins and therefore the dose received. In some embodiments with the use of nanoparticles, reading the fluorescence can be done by FACS.

In some embodiments:
a. the antibodies are fixed to a substrate so that each antibody is deposited in a known quantity at a coordinate x,y of the substrate so as to form a complexing unit or spot. For example, spots of antibodies directed against Ku70, phospho Ku70, H2AX or γ-H2AX, or any other antibody against proteins involved in the canonical ATM pathway. The substrate will comprise an antibody directed against a protein present in the sample, the quantity of which does not vary in the latter,
b. the antibodies organised in spots are then complexed with a protein extract in which the proteins are marked by fluorescence or colorimetry markers or any chemiluminescence or any other marker,
c. elimination of the non-complexed proteins, washing and rinsing of the blade, and
d. reading in a fluorescence or colorimetric scanner.

In some embodiments, the antibodies and the sample are complexed directly, after elimination of the non-complexed proteins and rinsing. The complex/sample whole is marked by fluorescent, colorimetric or chemiluminescent markers or any other marker capable of fixing on proteins, for example on the amine, carboxyl or peptide function of the proteins. After elimination of the markers that have not reacted, the antibody/protein complex is scanned and quantified. The quantity of fluorescence indicates the proportion of marked proteins and the concentration of antibodies. A non-complexed marked antibody spot makes it possible to determine the basic intensity for standardisation. In this embodiment, saturation of the substrate comprising the antibodies is carried out by means of a blocking substance not fixing the marking.

In some embodiments, the antibodies are fixed on nano- or microparticles, for example of polystyrene or diamond, optionally magnetised, such that a given antibody is fixed on a particle that is identifiable unequivocally, by a fluorescent marker, by a unique size, so that a given particle captures a given target. The targets marked with a fluorochrome different from the marking of the particles are put in contact with particles functionalised by the antibodies, after isolation and washing of the particles, the washed particles are analysed in FACS (preferably a portable microcapillary FACS). Measurement of the fluorescence specific to a particle or measurement of the diffraction qualifying the size of the particle makes it possible to identify the particle. Measurement of the fluorescence specific to the sample on each particle makes it possible to measure the quantity of target captured by the antibodies bound to the particle and makes it possible to deduce the abundance of the targets sought.

In some embodiments, for each target use is made of an antibody against the phosphorylated epitope, for example ENLEA(p)SGDYK (SEQ ID NO: 1) phospho Ku70 and another antibody directed against the non-phosphorylated epitope ENLEASGDYK (SEQ ID NO: 2) so that the phosphorylated proteins are captured solely by antibodies against the phosphorylated form and so that the non-phosphorylated form is captured solely by the antibody against the non-phosphorylated form. By means of this method, it is then possible to determine the proportion of phosphorylated form as I anti phospho Ku70/(1 anti phospho Ku70+I anti Ku70). The same couple is also developed for H2AX, γ-H2AX and all the other types of phosphorylated protein in the ATM cascade.

In some embodiments, the antibodies fixed on a particle or a substrate are complexed with a calibrated mixture (same concentration of proteins) consisting firstly of a sample to be analysed marked with a fluorescent, colorimetric, chemiluminescent, etc. marker: mixture A, and secondly a reference calibrated for the targets sought and marked with another fluorescent, colorimetric, chemiluminescent, etc. marker: mixture B. The two markers A and B emit different wavelengths that do not overlap. After complexing, the differently marked targets compete in order to bind with the complementary antibody. The ratio IA/IB of each target makes it possible to quantify the target A, the concentration of the reference target B being known.

In some embodiments, references come from extracts of lymphocytes and/or of hair follicle cells and/or keratinocyte and/or immortalised cell irradiated in various doses. Each reference point is irradiated at a precise dose, and all the reference points form a range. Each point in the range is complexed and analysed in the same way as the other extracts.

In some embodiments, the antibody/antigen complexification is analysed in a portable scanner. This scanner will comprise a matrix of LEDs emitting at an excitation wavelength for marking the probes and/or targets such that an LED is disposed below each housing of a plate with 96 wells or 384 wells and more generally N wells. The bottoms of the wells of the 96-well or N-well plates are made from a material that is transparent to the wavelength of the LEDs. When various markings are used, one LED for each type of excitation is disposed under each housing. Above the 96-well or N-well plate an optical filter is arranged, filtering the excitation of wavelengths coming from the LEDs and transparent to the emission wavelength of the markings. The whole is disposed in a dark room surmounted by a sensor, camera, CCD, digital photographic apparatus and silver apparatus, with a device for focusing on the bottom of the wells and/or on the surface of the plates. The sensor makes it possible to produce a photograph of the N-well plate. Accumulating the signal makes it possible to determine the degree of complexing of each target. In some embodiments, the filter is arranged in front of the sensor.

In some embodiments, the antibodies will be replaced by Fab-zipper chimeric molecules described in the application FR-A1-2 958 645. The chimeric molecules are marked or fixed on a substrate preferentially by means of a nucleic acid complementary or nucleic acid analogue sequence fixed on the substrate or marked by a chromophore.

The sequences used for creating the phosphorylated anti-Ku740 Fab-zipper on serine 27 are Light chain:

(SEQ ID 3)
MMSPAQFLFLLVLWIRVSETIGDVVMTQTPLTLSVTFGQPASISCKS
SQSLLDRDGKTYLNWLLQRPGQSPKRLIYLVSKLDSGVPDRFTGSGS
GTDFTLKISRVEAEDLGVFYCWQGTHLPQTFGGGTKLEVKRADAAPT
VSIFPPSSEQLTSGGASVVCFLNNFYPKDINVKWKIDGSERQNGVLN
SWTDQDSKDSTYSMSSTLTLTKDEYERHNSYTCEATHKTSTDQDSKD
STYSMSSTLTLTKDEYERHNSYTCEATHKTSTSPIVKSFNRNEC

Heavy Chain:

(SEQ ID 4)
MELGLSWVFLVALLNGVQCQVQLVETGGGLVRPGNSLKLSCVTSGFT
FSKYRMHWLRQFPGKRLEWIAAITVKSDNYGAHYVESVKGRFTISRD
DSKSSVYLQMNRLREEDTATYYCCTTGFAYWGQGTLVTVSAAKTTPP
SVYPLAPGSAAQTNSMVTLGCLVKGYFPEPVTVTWNSGSLSSGVHTF
PAVLQSDLYTLSSSVTVPSSTWPSETVTCNVAHPASSTKVVLQSDLY
TLSSSVTVPSSTWPSETVTCNVAHPASSTKVDKKIVPRDCGCKPCIC
TVPEVSSVFIFPPKPKDVLTITEEQFNSTFRSVSELPIMHQDWLNGK
EFKCRVNSAAFPAPIEKTISKTKGRPKAPQVYTIPPPKEQMAKDKVS
LTCMITDFFPEDITVEWQWNGQPAENYKNTQPIMDTDGSYFVYSKLN
VQKSNWEAGNTFTC

Alternative Heavy Chain:

(SEQ ID 5)
MELGLSWVFLVALLNGVQCQVQLVETGGGLVRPGNSLKLSCVTSGFT
FSKYRMHWLRQFPGKRLEWIAAITVKSDNYGAHYVESVKGRFTISRD
DSKSSVYLQMNRLREEDTATYYCCTTGFAYWGQGTLVTVSAAKTTPP
SVYPLAPGSAAQTNSMVTLGCLVKGYFPEPVTVTWNSGSLSSGVHTF
PAVLQSDLYTLSSSVTVPSSTWPSETVTCNVAHPASSTKVVLQSDLY
TLSSSVTVPSSTWPSETVTCNVAHPASSTKVDKKIVPRDCGCKPC

A peptide, LPXTG, is fused as the C-terminus of each of the sequences. The peptide LPXTG fused to one of the heavy or light chains is itself fused as a C-terminus at the 5-terminal end (N′) or respectively the 3-terminal end (C′) of a sequence of nucleic acids (DNA) or analogues, such that the nucleic acid sequences are complementary. By hybridising, they restructure the active site of the antibody.

FIG. 31 shows the results of the fluorescence intensity from γ-H2AX from cells irradiated at 0, 2 and 8 Gy (Millipore), respectively of a γ-H2AX antibody and a γ-H2AX Fab-zipper.

In some embodiments, the chimeric molecule has an addressing marker made from a nucleic acid and a nucleic acid analogue such that the marker allows hybridisation at a precise position x,y at the bottom of each well. The complementary marker makes it possible to functionalise the bottom of the wells. In some embodiments, the Fab-zippers having a marker are complexed with the extract to be analysed and then the whole is disposed in a well in the plate so that the marker can allow addressing of the Fab-zippers complexed to the target at the position of the marker.

In some embodiments, nano- or microparticles are provided with a marker for functionalising the particles by Fab-zipper comprising a complementary marker. In other embodiments, the particles comprise a second marker for addressing them in the bottom of a 96- or N-well plate. In other embodiments, the second marker makes it possible to mark the nanoparticle with a particular fluorescent nucleic acid.

In some embodiments, the glue patches are disposed on the body of an individual (FIG. 32), for example with a label-dispensing device, so that the patches are disposed on a dispensing strip and so that the patch of glue is provided with a label comprising signs for identifying the label unequivocally. For example, a bar code or a succession of geometric signs makes it possible to identify the patch unequivocally. In particular a label is composed of 4 geometric signs, triangle, circle, square, arranged in 5 successive positions. For example, the first position indicates the direction of reading, for example, a triangle with the vertex in the mediant position. The following four positions are a combination of triangle top and bottom, circle, square, this making it possible to code up to 256 cases. An additional position makes it possible to code 1024 cases. The use of an additional character makes it possible to code 626 cases on four positions and 3125 cases on five positions. The use of a camera of the Kinet® type and an image analysis makes it possible to recognise the body location of the patches and to identify the positioning thereof on the 96- or N-well plate. A patch may have a width slightly greater than that of a well and a length slightly greater than that of three consecutive wells, so that the hairs attached to this patch are substantially distributed in three consecutive wells.

An example in which hydrogenated nanodiamonds (H-NDs) are used as a substrate for fixing proteins. The nanodiamonds have a diameter of between 1 and 100 nm. In a variant, it would be possible to use microdiamonds (1 μm-999 μm) or submicron diamonds (100 nm-700 nm). In a variant, hydrogenated activated carbon could be used.

H-NDs have been used as objects for capturing a mixture of proteins coming from two extraction methods: a conventional one (lysis buffer) and another one based on microwaves (15 seconds at 450 W). ZR75.1 human cells, established in the laboratory, were used as a cell model since they have a strong phospho-Ku70 induction after irradiation. The cells were irradiated from a Cs¹³⁷ source (IBM637, CisBio International, Gif sur Yvette, France) at a dose of 10 Gy. One hour after irradiation, the irradiated and non-irradiated cells were recovered and counted so as to obtain 1 million cells per condition (per tube). Extraction was then carried out on both the irradiated and the non-irradiated cells. Then 100 μg of H-NDs was added to the protein extract and the whole was incubated for 10 minutes at ambient temperature and under continuous agitation so as to capture the proteins on the surface of the H-NDs. Then the tubes were centrifuged for 5 minutes at 1500 revolutions per minute in order to recover the H-NDs. A PBS rinsing was subsequently carried out (centrifugation for 5 minutes at 1500 revolutions per minute), and then the residues were put in contact with a mixture of anti-phospho-Ku70 primary antibody (1/1000) and Alexa Fluor 488 secondary antibody (1/500) for 15 minutes at 37° C. and under continuous agitation. The tubes were next centrifuged for 5 minutes at 1500 revolutions per minute in order to recover the H-NDs and to eliminate the excess primary antibody. Two PBS rinsings were subsequently carried out (centrifugation for 5 minutes at 1500 revolutions per minute), then the residues were taken up in 250 μl of PBS in order to pass the samples over an FACSCalibur™ using Cell Quest Pro software during the acquisitions. As for the fluorescence of the Alexa Fluor 488, this was detected on the FL1 channel of the cytometer. Precipitations by centrifugation can be replaced by magnetic precipitations in 30 seconds with magnetic H-NDs comprising iron crystals.

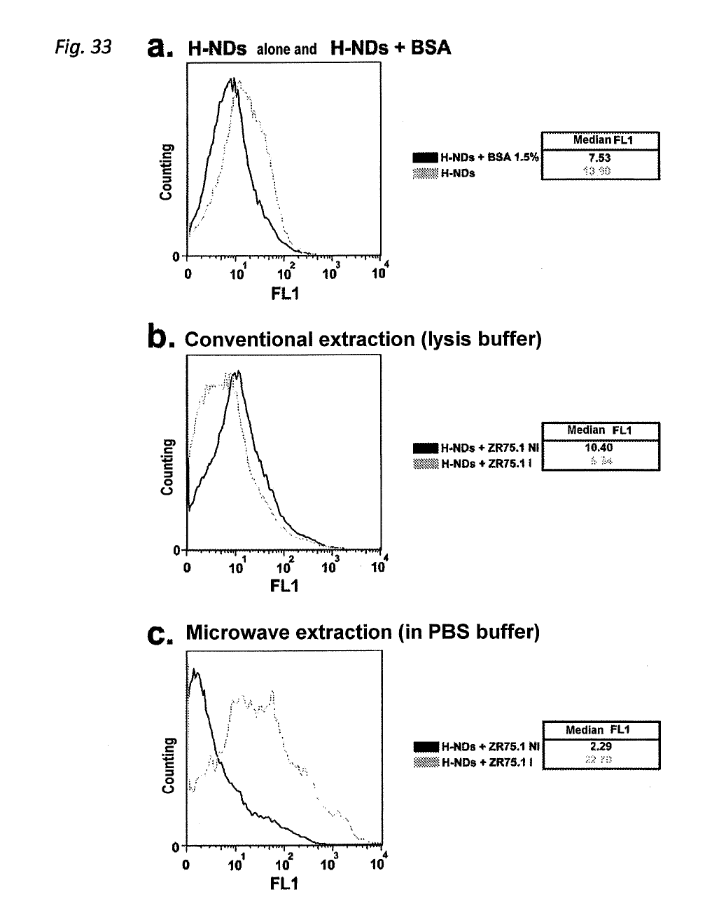

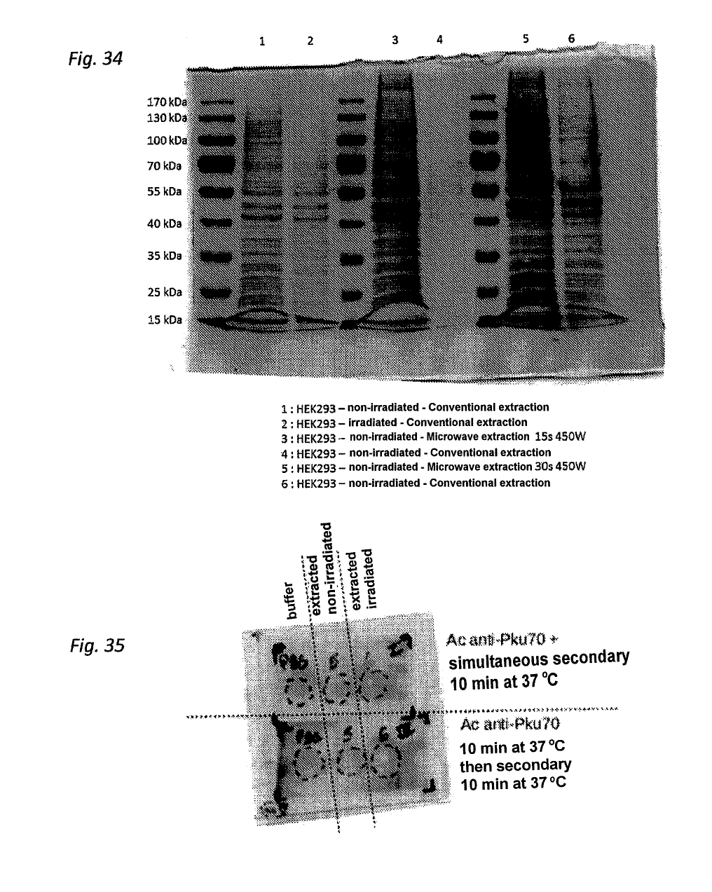

FIGS. 33a, b and c illustrate the results obtained on the change in the fluorescence signal (FL) of an anti Ku70 marking in FACS for protein extracts of human cells (ZR75.1) irradiated at 10 Gy (grey) or not (black), and captured on hydrogenated nanodiamond particles, with various extraction modes.

FIG. 35 illustrates the implementation of a rapid transfer of the dot blot type for samples irradiated at 0.5 Gy, on membranes. The depositions are carried out on nitrocellulose or PVDF membranes and dried (maximum 2 minutes at 37°).

The membranes are immersed in a saturation bath (1×TBS-3% BSA) under agitation for 5 minutes.

The membranes are next immersed in a bath containing a mixture of primary and secondary antibodies to the correct dilutions (according to the technical data notes for the antibodies) in saturation solution under agitation at 37° C. for 10 minutes. It is clear that the mixture of primary and secondary antibodies gives a result in 10 minutes similar to the sequential use of the two antibodies over a period of 20 minutes.

Finally, the membranes are rinsed with 1×TBS and revealed according to the type of marking of the secondary antibody (ECL or fluorescence).

An example of a quantification method.

The quantification method consists of carrying out at least two measurements of the phosphorylation of proteins of the ATM cascade. The at least two measurements are done for example on the same protein at at least two different times, or on two proteins at the same time, or comprises any combination of measurements at different times on different proteins.

The measurements are transferred onto standard reference curves or surfaces forming response dose ranges.

Following irradiation, the phosphorylation/dephosphorylation kinetics of two ATM proteins are such that the degree of phosphorylation, as a function of time (t), for example H2AX(t)=Iₕ, and Ku70(t)=Iₖ, increase initially at rates that may be different for the two proteins, and secondly decrease at rates that may be different for the two proteins. The kinetics goes to its maximum at times T_{H2AX} and T_{Ku70}, which may be different.

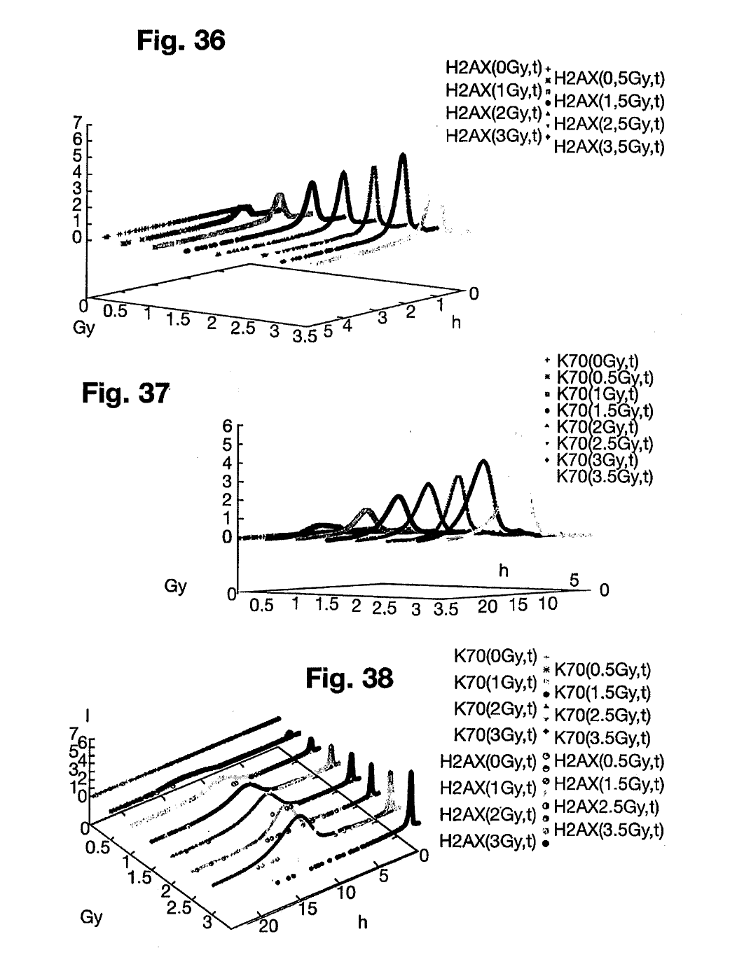

For example, H2AX(t) goes to its maximum typically on average one hour after irradiation whereas Ku70 goes to its maximum typically on average 12 hours after irradiation (cf. FIG. 36-38).

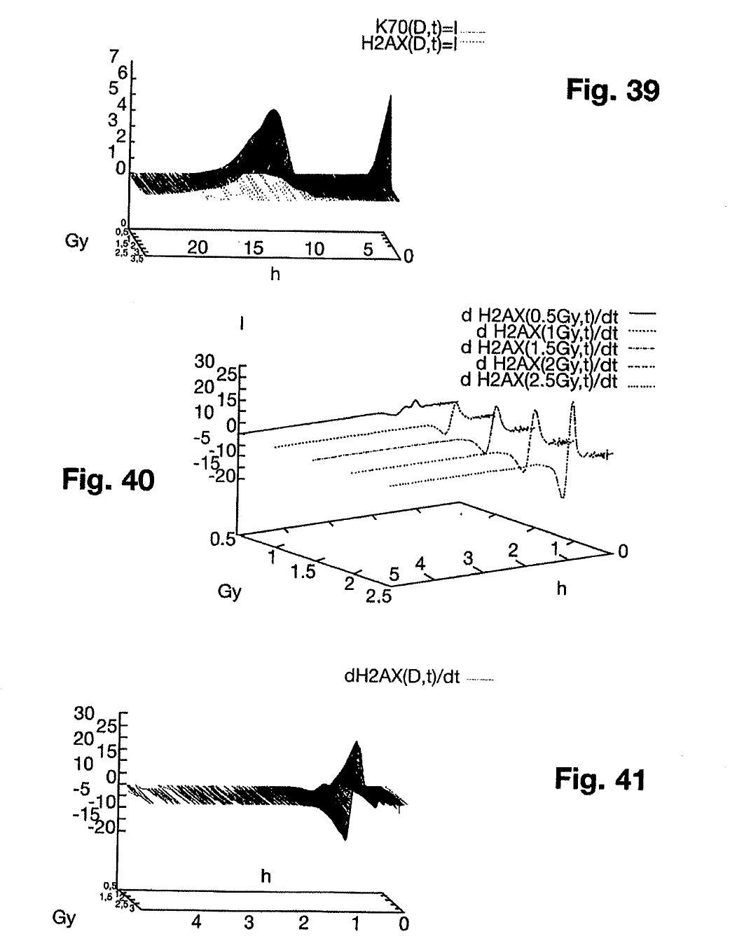

Thus, observing the phosphorylation/dephosphorylation kinetics as a function of the irradiation dose (D) of two ATM proteins such as H2AX and Ku70 shows a dependency defined as H2AX(D)=Iₕ and Ku70(D)=Iₖ, thus defining a surface in three-dimensional space (D, t, I) where a coordinate I corresponds to each coordinate D and t for each of the proteins so that: H2AX(D,t)=Iₕ and Ku70(D,t)=Iₖ (cf. FIG. 39).

These surfaces represent standard references or ranges for determining the degree of irradiation of an individual from a measurement. They make it possible, from the isolated measurement i made at the same moment for two ATM proteins having different phosphorylation kinetics, to solve the equation system:

H2AX(D,t)=iₕ,

Ku70(D,t)=iₖ,

and to determine the dose received and the moment t₀ of irradiation.

In some cases, the use of one or two proteins does not make it possible to converge towards a single dose/time solution. A large number of proteins in the ATM system may then be examined.

Nevertheless, in another embodiment, the curves of

H2AX(t)=Iₕ,

H2AX(D)=Iₕ,

Ku70(t)=Iₖ,

Ku70(D)=Iₖ,

and the surfaces H2AX(D,t)=Iₕ and Ku70(D,t)=Iₖ may be derived as a function of the time and/or dose so that d H2AX(t)/dt=I′ₕ, d Ku70(t)/dt=I′ₖ. Likewise, the surface may be derived as a function of time so that d H2AX(D,t)/dt=I′ₕ, d Ku70(D,t)/dt=I′ₖ (cf. FIG. 40-41).

For example, by measuring Ku70(D,t)=iₖ₍ₜ₎ at a given time (t) and Ku70(D,(t+Δt))=i_{k(t+Δt)} with a delay t+Δt, it is possible to evaluate, at a singular point, d Ku70(D,t)/dt=i′ₖ by [Ku70(D,(t+Δt))−K70(D,ti)]/Δt=(i_{k(t+Δt)}−iₖ₍ₜ₎₎/Δt. Likewise, by measuring H2AX(D,t)=iₕ₍ₜ₎ at a given time (t) and H2AX(D,(t+Δt))=i_{h(t+Δt)} with a delay t+Δt, it is possible to evaluate, at a singular point, d H2AX(D,t)/dt=i′ₕ by [H2AX(D,(t+Δt))−H2AX(D,ti)]/Δt=(i_{h(t+Δt)}−i_{h (t)})/Δt.

Thus, by having the surface

Ku70(D,t)=Iₖ,

H2AX(D,t)=Iₕ,

and derived surfaces

d Ku70(D,t)/dt=I′ₖ,

d H2AX(D,t)/dt=I′ₕ,

it is possible to solve the equation system

d Ku70(D,t)/dt=(i_{k(t+Δt)}−iₖ₍ₜ₎)/Δt,

Ku70(D,t)=iₖ,

d H2AX(D,t)/dt=(i_{h(t+Δt)}−iₕ₍ₜ₎)/Δt,

H2AX(D,t)=iₕ,

and to overcome any uncertainties with a single solution determining the dose received and the moment of irradiation from the observation of the degree of phosphorylation of a protein and the variation in this degree in a short period of time.

The precision of observation will be improved by observing at the same time several proteins such as preferably proteins having a rapid response such as H2AX with a maximum response for example around 1 hour and Ku70 with a maximum response for example around 12 hours.

1. Method for evaluating, via biodosimetry, the irradiation dose received by a person subjected to ionising radiation, comprising the steps consisting of:
a) sampling, in at least one region of the body of the individual, hair bulbs or follicles of bristles and/or hairs of the individual,
b) extracting proteins from cells of these hair bulbs or follicle sampled, these proteins comprising proteins from the ATM system that have undergone phosphorylation and/or acetylation caused by the ionising radiation, and
c) analysing at least two types of protein extracted and interpreting the analysis results in order to determine the irradiation dose received in the or each sampling region, the phosphorylated and/or acetylated proteins from the ATM system being intended to produce signals during the analysis, a quantity of which makes it possible to evaluate this irradiation dose.

2. Method according to claim 1, wherein said at least two types of protein have different phosphorylation/dephosphorylation kinetics.

3. Method according to claim 1, wherein the proteins are immobilised on a substrate at step c), the proteins being fixed directly or indirectly on said substrate.

4. Method according to claim 1, wherein at least the proteins H2AX and Ku70 are analysed at step c).

5. Method according to claim 1, wherein the hair follicles or hair bulbs and/or hairs are sampled in several distinct regions of the body so as to determine the irradiation dose received by each region.

6. Method according to claim 1, wherein the sampling step a) comprises the substeps consisting of:
applying a patch to the or each region, and removing it in order to pull off bristles and/or hairs with their hair follicles or bulbs in this region, and
introducing the patch and bristles and/or hairs with their hair follicles or bulbs into a reaction well.

7. Method according to claim 6, wherein the patch is fixed to one end of a piston able to move in a tube between an advanced position and a retracted position, in the advanced position the patch extending at least partly outside the tube and being able to be applied by means of the piston to the region, and, in the retracted position, the patch being housed in the tube, removing the patch from the region causing the bristles and/or the hairs to be pulled off with their hair follicles or bulbs, and retraction of the piston into the tube causing the bristles and/or hairs with their hair follicles or bulbs attached to the patch to be introduced into the tube.

8. Method according to claim 6, wherein the patch is formed by a flexible strip that is fixed by one end to a rod carried by a plug, the strip being intended to be coiled around the rod before it is introduced into the reaction well.

9. Method according to claim 7, wherein the patch is introduced into the well by fitting an open end of the tube in the well or inserting the rod and the strip in the well and then closing this well with the plug carrying the rod.

10. Method according to claim 1, wherein it further comprises the steps consisting of:
taking blood from the individual, for example at the end of a finger,
recovering cells from the blood taken, with the exception of the red corpuscles, and extracting proteins from these cells, these proteins comprising proteins of the ATM system that underwent phosphorylation and/or acetylation caused by the ionising radiation.

11. Method according to claim 10, wherein the sampling step is carried out by means of a lancet intended to pierce the skin of the individual, this lancet comprising an ejectable blade and preferably comprising a chamber for storing a solution of alcohol and heparin, which is intended to be released at the time of ejection of the blade.

12. Method according to claim 10, wherein the cell recovery step consists of:
introducing the sampled blood into a tube containing a suspension of magnetic balls functionalised with antibodies against blood group antigens, and optionally heparin,
separating the balls from a residual liquid by means of a magnetic field, the residual liquid comprising the blood cells, with the exception of the red corpuscles, and transferring the residual liquid into a reaction well.

13. Method according to claim 1, wherein the extraction of the proteins comprises the substeps consisting of:
putting the sampled hair follicles and/or the recovered residual liquid in contact with, or mixing them with, a cell lysis solution, or
putting the cells in contact with, or mixing them with, a non-denaturing solution, preferably free from phosphorus, and then subjecting the whole to a mechanical energy, or by microwave or ultrasound, intended to cause lysis of the cells.

14. Method according to claim 1, wherein various samples are distributed in various reaction wells of a multiwell plate, and in that the bottom of the wells is formed by a membrane, optionally a filtering membrane, on which the proteins are intended to be deposited or fixed, directly or by means of specific antibodies previously fixed to the bottom or to particles, this bottom preferably being removable.

15. Method according to claim 14, wherein markers (are introduced into the reaction wells, these markers being fluorescent, colorimetric or chemiluminescent markers, and/or antibodies, said markers being intended to bind to phosphorylated and/or non-phosphorylated proteins.

16. Method according to claim 15, wherein the markers are fixed to particles, each type of marker being for example fixed to a particle of given size that is different from the sizes of the particles carrying the other types of marker.

17. Method according to claim 1, wherein the analysis and interpretation step c) comprises the substeps consisting of:
subjecting each type of protein to a marking, the phosphorylated form of each type of protein optionally being able to have a specific marking,
subjecting the proteins to an analysis in which at least one signal caused by the marking of the protein type is studied,
comparing a quantity or a parameter of this signal with a calibration curve representing the change in this quantity as a function of an irradiation dose received by an individual, and
deducing therefrom the irradiation dose received by the individual, in the sampling region.

18. Method according to claim 17, wherein the proteins are analysed:
by an LIBS method that makes it possible to quantify at least the phosphorylation, each type of protein, in its phosphorylated and non-phosphorylated forms, optionally being able to be subjected to a marking, for example with boron, or
by fluorescence, colorimetry or chemiluminescence, each type of protein having been subjected to a fluorescent, colorimetric or chemiluminescent marking.

19. Method according to claim 17, characterised in that a predetermined quantity of radiomimetic chemotherapeutic substance is added to the proteins extracted, the parameter studied being:
IP: the intensity of at least one signal corresponding to the phosphorylated form of one of each type of protein, and/or
IP/I, the ratio between the intensity IP and the intensity I, I being the intensity of at least one signal corresponding to the marking of each type of protein, in its phosphorylated and non-phosphorylated forms, and/or
IP/IP_{treated}: the ratio between the intensity IP and the intensity IP_{treated}, IP_{treated} being the intensity of at least one signal corresponding to the phosphorylated and treated form of each type of protein, and/or
IP_{treated}/I_{treated}: the ratio between the intensity IP_{treated} and the intensity I_{treated}, I_{treated} being the intensity of the signal corresponding to the marking of each type of treated protein, in its phosphorylated and non-phosphorylated forms, and/or
(IP/I)/(IP_{treated}/I_{treated}): the ratio between the intensities IP and I and the ratio between the intensities IP_{treated} and I_{treated}.

20. Method according to claim 17, wherein step a) consists of sampling hair follicles or hair bulbs and/or hairs on at least two occasions in the same sampling region.

21. Method according to claim 20, wherein the samplings are carried out at a predetermined interval of time Δt or are carried out almost simultaneously and at least one of the samples is cultured for a given period Δt.

22. Method according to claim 21, wherein the parameter studied is:
IP: the intensity of at least one signal corresponding to the phosphorylated form of one of each type of protein, and/or
IP/I: the ratio between the intensity IP and the intensity I, and/or
dIP/dt: the variation over time in the intensity IP, and/or
d(IP/I)/dt: the variation over time in the ratio IP/I.

23. Method according to claim 20, wherein two or more types of protein each comprise a marking that is particular to them so that these types of protein can be identified by their markings and by the properties of these markings, the phosphorylated forms of these types of protein also being able to be marked.

24. Method according to claim 20, wherein at least two of the samples are mixed with a radiomimetic chemotherapeutic substance at a known dose, one of the mixtures being analysed immediately and the other being analysed after a given period, the parameter studied being:
IP_{treated}: the intensity of at least one signal corresponding to the phosphorylated and treated form of each type of protein, and/or
IP_{treated}/I_{treated}: the ratio between the intensity IP_{treated} and the intensity I_{treated}, I_{treated} being the intensity of the signal corresponding to the marking of each type of treated protein, in its phosphorylated and non-phosphorylated forms, and/or
dIP_{treated}/dt: the variation over time in the intensity IP_{treated}, and/or
d(IP_{treated}/I_{treated})/dt: the variation over time in the ratio IP_{treated}/I_{treated}.

25. Method according to claim 1, wherein it is applied to a plurality of individuals, in a place not normally equipped with laboratory apparatus.

26. Method according to the preceding claim, wherein it comprises a preliminary step of identifying the or each individual.

27. Method according to claim 1, in which the irradiation dose or the moment of irradiation is determined by means of a surface setting out the variation in the degree of phosphorylation of at least one protein as a function of the irradiation dose received and the observation delay time.

28. Method according to the preceding claim, in which the irradiation dose or the moment of irradiation is determined by means of a derivative or a finite increment of said surface.

29. Kit for implementing the method according to claim 1, wherein it comprises at least one device for sampling bristles and/or hairs of an individual, optionally a device for sampling the blood of an individual, and a multiwell plate, the patches used for sampling the bristles and/or hairs being intended to be introduced into different wells in the multiwell plate.
